(19) United States
(12) Patent Application Publication
(10) Pub. No.: US 2026/0283779 A1
(43) Pub. Date: Sep. 24, 2026

(54) Systems and Methods for an Endoluminal Prosthesis having a Constrictive Sleeve Member

(71) Applicant: Angiomed GmbH & Co. Medizintechnik KG, Karlsruhe (DE)

(72) Inventors: Amal Belkouch, Karlsruhe (DE); Mathew Dixon, Mesa, AZ (US); Carsten Giebmeyer, Karlsruhe (DE)

(21) Appl. No.: 19/660,223

(22) Filed: Apr. 27, 2026

Related U.S. Application Data
(63) Continuation-in-part of application No. PCT/US2024/057178, filed on Nov. 22, 2024.
(63) Continuation-in-part of application No. PCT/US2024/029286, filed on May 14, 2024.

(51) Int. Cl. A61F 2/07; A61F 2/958

(52) U.S. Cl. CPC A61F 2/07; A61F 2/958; A61F 2002/072; A61F 2210/0014; A61F 2220/0033; A61F 2220/0058; A61F 2230/0004; A61F 2230/0091; A61F 2250/0037; A61F 2250/0039; A61F 2250/0098

(57) ABSTRACT

A stent graft including a framework with a helical section, a woven section, and a transitional section between the helical section and the woven section. The stent graft can include a plurality of radiopaque markers distributed about an end portion of the helical section and/or an end portion opposite the transitional section. The graft of the stent graft can include an inner graft member and an outer graft member encapsulating at least the helical section of the framework. The stent graft can further include a constrictive sleeve over the outer graft member, the constrictive sleeve configured to resist radial expansion of the stent graft over a length of the stent graft commensurate with the constrictive sleeve.

### PRIORITY

This application is a U.S. bypass continuation-in-part application claiming priority to International Application No. PCT/US2024/057178, filed Nov. 22, 2024, which is a continuation-in-part of International Application No. PCT/US2024/029286, filed May 14, 2024, each of which is incorporated by reference in its entirety into this application.

### BACKGROUND

In a healthy person, blood flowing from the stomach, esophagus, or intestines first flows through the liver. In an unhealthy person having, for example, liver damage, there can be blood flow-restricting blockages such that blood cannot easily flow through the liver. Such a condition is known as portal hypertension. Common causes of portal hypertension include alcohol abuse, blood clots in a vein that flows from the liver to the heart, too much iron in the liver (e.g., hemochromatosis), hepatitis B, or hepatitis C. When portal hypertension occurs, the blood flow-restricting blockages can elevate pressure in the portal vein causing it to rupture and seriously bleed. A person with portal hypertension can also have bleeding from the veins of the stomach, esophagus, or intestines (e.g., variceal bleeding), a buildup of fluid in the belly (e.g., ascites), or a buildup of fluid in the chest (e.g., hydrothorax).

While endoluminal prosthesis are known in the art, there are also known problems with current endoluminal prosthesis. For example, deployment of an endoluminal prosthesis may include the positioning of the endoluminal prosthesis within a patient's vasculature at a point where a blockage restricts blood flow and be self-expanding to open the patient vasculature and enable blood flow through the endoluminal prosthesis. Some endoluminal prosthesis may include a radially restraining sleeve member, or more generally, a sleeve or sleeve member, disposed around a middle portion of the endoluminal prosthesis to restrict the expansion of the endoluminal prosthesis to a predefined maximum diameter. Interference with the expansion of the endoluminal prosthesis may arise when the sleeve is continuously attached, e.g., sintered, to the stent graft along the entire length of the sleeve. As a result, most sleeves are not sintered to the stent graft and the slippage of the sleeve that occurs during expansion of the endoluminal prosthesis has typically been accepted by medical professionals. Disclosed herein are embodiments of an endoluminal prosthesis configured to obviate the slippage of the sleeve on an endoluminal prosthesis for treating at least portal hypertension.

### SUMMARY

Disclosed herein is an endoluminal prosthesis including, in some embodiments, a stent graft including a stent and a base graft secured to the stent, wherein the stent graft is expandable in a radial direction of the endoluminal prosthesis and exhibiting a self-expansion force in the radial direction, wherein the stent graft is configured to self-expand in the radial direction from a compacted condition to a self-expanded condition, and wherein the stent graft is adjustable in a range between the self-expanded condition and a maximum expanded condition beyond the self-expanded condition by application of an external force. The endoluminal prosthesis may further include a radially restraining sleeve member having first and second ends and a middle portion, wherein each of the first and second ends are fixedly coupled to the stent graft through a heat application process. The radially restraining sleeve member may be adjustable to an adjusted diameter in the radial direction in a range of diameters between the self-expanded condition of the stent graft and the maximum expanded condition of the stent graft through the application of an external force.

The endoluminal prosthesis may be an endovascular prosthesis. In some embodiments, the radially restraining sleeve member may be made from expanded polytetrafluorethylene (ePTFE). In some instances, the base graft covers the stent both on the inner, lumen-facing, side and on the outer, vessel-facing, side of the endoluminal prosthesis. In various embodiments, the heat application includes exposure of the first and second ends of the radially restraining sleeve member to heat within a temperature range of 180-200° C. at a pressure of approximately 20 bar (290 PSI). In some particular embodiments, the heat has a temperature of 327° C. when material surfaces are in tight contact.

In various examples, the heat application includes exposure of the first and second ends of the radially restraining sleeve member to heat around an entire circumference of the radially restraining sleeve member. In yet further embodiments, the heat application includes exposure of the first and second ends of the radially restraining sleeve member to heat at a plurality of discrete points around a circumference of the radially restraining sleeve member. In some instances, the radially restraining sleeve member is fixedly coupled to the stent graft through the heat application process at one or more additional discrete points between the first and second ends of the radially restraining sleeve member. Further, the radially restraining sleeve member may include a middle portion between the first and second ends, and wherein the middle portion is slidably disposed on the stent graft.

Additionally, disclosed herein is a method for treating a lumen of a subject with an endoluminal prosthesis, the endoluminal prosthesis including a stent graft including a stent and a base graft secured to the stent, wherein the stent graft is expandable in a radial direction of the endoluminal prosthesis and exhibiting a self-expansion force in the radial direction, wherein the stent graft being configured to self-expand in the radial direction from a compacted condition to a self-expanded condition, and wherein the stent graft being adjustable in a range between the self-expanded condition and a maximum expanded condition beyond the self-expanded condition by application of an external force, and a radially restraining sleeve member having first and second ends and a middle portion, wherein each of the first and second ends are fixedly coupled to the stent graft through a heat application process. In some embodiments, the radially restraining sleeve member is adjustable to an adjusted diameter in the radial direction in a range of diameters between the self-expanded condition of the stent graft and the maximum expanded condition of the stent graft through the application of an external force.

The method may include the steps of introducing the endoluminal prosthesis in a compacted condition into a lumen by means of a delivery device; advancing the endoluminal prosthesis towards a target location by means of the delivery device; releasing the endoluminal prosthesis at a target location such that the endoluminal prosthesis self-expands from the compacted condition to a self-expanded condition; applying an external force on the endoluminal prosthesis in a radial direction thereof such that the endoluminal prosthesis further expands from the self-expanded condition to an expanded condition between the self-expanded condition and a maximum expanded condition; and releasing the external force from the endoluminal prosthesis. After releasing the external force from the endoluminal prosthesis, the endoluminal prosthesis is configured to maintain the expanded condition. In some embodiments, the method may further include performing a post-implantation adjustment to the diameter of the endoluminal prosthesis for a patient in need of such adjustment.

Also disclosed herein is a stent graft including, in some embodiments, a framework, a plurality of radiopaque markers, a graft, and a constrictive sleeve. The framework includes a helical section, a woven section, and a transitional section between the helical section and the woven section. The radiopaque markers are distributed about an end portion of the helical section opposite the transitional section, an end portion of the woven section opposite the transitional section, or both the end portion of the helical section and the end portion of the woven section. The graft includes an inner graft member and an outer graft member encapsulating at least the helical section of the framework. The constrictive sleeve is over the outer graft member of the graft. Such a constrictive sleeve is configured to resist radial expansion of the stent graft over a length of the stent graft commensurate with the constrictive sleeve.

In some embodiments, the framework consists of a single wire and three wire welds. The wire is formed into each section of the helical section, the woven section, and the transitional section of the framework. The three wire welds are respectively distributed about the helical section, the woven section, and the transitional section of the framework to maintain a topology of the framework.

In some embodiments, an end portion of the wire in the end portion of the helical section opposite the transitional section is welded to an internal portion of the wire in the end portion of the helical section of the framework.

In some embodiments, the end portion of the wire in the end portion of the helical section opposite the transitional section of the framework includes a rounded end of the wire, thereby obviating any potentially traumatic edges in the end portion of the helical section opposite the transitional section.

In some embodiments, an end portion of the wire in the transitional section is welded to an internal portion of the wire in the transitional section of the framework.

In some embodiments, the end portion of the wire in the transitional section of the framework includes a rounded end of the wire, thereby obviating any potentially traumatic edges in the transitional section.

In some embodiments, a looped portion of the wire in the end portion of the woven section opposite the transitional section is welded to an internal portion of the wire in the end portion of the woven section of the framework.

In some embodiments, the wire consists essentially of nitinol.

In some embodiments, the end portion of the helical section opposite the transitional section of the framework includes three radiopaque markers crimped onto the framework approximately equidistant from each other around the end portion of the helical section.

In some embodiments, the end portion of the woven section opposite the transitional section of the framework includes three radiopaque markers crimped onto the framework approximately equidistant from each other around the end portion of the helical section.

In some embodiments, the radiopaque markers consist essentially of tantalum.

In some embodiments, the stent graft further includes a radiopaque marker band around the transitional section of the framework. The radiopaque marker band consists essentially of tungsten, which is printed as discrete quadrilaterals on a ring member disposed between the framework and the outer graft member of the graft.

In some embodiments, each graft member of the inner graft member and the outer graft member independently consists essentially of ePTFE, optionally, impregnated with carbon for reduced thrombogenicity.

In some embodiments, the inner graft member consists essentially of ePTFE impregnated with carbon. However, the outer graft member consists essentially of ePTFE without carbon impregnated therein.

In some embodiments, the constrictive sleeve is coupled to the graft exclusively by an interference fit.

In some embodiments, the constrictive sleeve has a length shorter than that of the graft. The constrictive sleeve is approximately centered over the graft. Both the end portion of the helical section opposite the transitional section and an end portion of the helical section proximate the transitional section extending through the end portion of the woven section opposite the transitional section are flared in a ready-to-deploy state of the stent graft.

In some embodiments, the constrictive sleeve includes a tube consisting essentially of ePTFE and an inner membrane and an outer membrane consisting essentially of ePTFE over the tube.

In some embodiments, the inner membrane, the outer membrane, or both the inner membrane and the outer membrane have a greater resistance to radial expansion than the tube.

In some embodiments, the constrictive sleeve is configured to resist radial expansion of the stent graft over the length of the stent graft commensurate with the constrictive sleeve until radially expanded in vivo with an elongate balloon.

Also disclosed herein is a stent graft including, in some embodiments, a framework, a plurality of tantalum markers, a graft, a tungsten radiopaque marker band, and a constrictive sleeve. The framework includes a helical section, a woven section, and a transitional section between the helical section and the woven section. Such a framework consists of a single nitinol wire and three wire welds. The wire is formed into each section of the helical section, the woven section, and the transitional section of the framework. The three wire welds are respectively distributed about the helical section, the woven section, and the transitional section of the framework to maintain a topology of the framework. Three tantalum markers are crimped onto the framework approximately equidistant from each other around an end portion of the helical section opposite the transitional section of the framework. Another three tantalum markers are crimped onto the framework approximately equidistant from each other around an end portion of the woven section opposite the transitional section of the framework. The graft includes an inner graft member and an outer graft member encapsulating at least the helical section of the framework. The inner graft member consists essentially of ePTFE impregnated with carbon for reduced thrombogenicity. However, the outer graft member consists essentially of ePTFE without carbon impregnated therein. The tungsten radiopaque marker band is around the transitional section of the framework. Such a tungsten radiopaque marker band is printed as discrete squares on a ring member disposed between the framework and the outer graft member of the graft. The constrictive sleeve is shorter in length than the graft, approximately centered over the graft, and coupled to the outer graft member of the graft exclusively by an interference fit. The constrictive sleeve includes a tube consisting essentially of ePTFE and an inner membrane and an outer membrane consisting essentially of ePTFE over the tube. The constrictive sleeve is configured to resist radial expansion of the stent graft over the length of the stent graft commensurate with the constrictive sleeve until radially expanded in vivo with an elongate balloon.

These and other features of the concepts provided herein will become more apparent to those of skill in the art in view of the accompanying drawings and following description, which describe particular embodiments of such concepts in greater detail.

### BRIEF DESCRIPTION OF THE DRAWINGS

One or more embodiments of the present disclosure are illustrated by way of example and not limitation in the figures of the accompanying drawings, in which like references indicate similar elements.

FIG. 1 illustrates an endoluminal prosthesis in a portal vein in accordance with some embodiments.

FIG. 2A illustrates a side-on view of an endoluminal prosthesis in accordance with some embodiments.

FIG. 2B illustrates a detailed view of the endoluminal prosthesis of FIG. 2A about a coupling between a terminal frame and an annular member of a main frame of the endoluminal prosthesis in accordance with some embodiments.

FIG. 3A illustrates a side-on view of the endoluminal prosthesis of FIG. 2A having a first sleeve disposed thereon in accordance with some embodiments.

FIG. 3B illustrates a detailed view of the endoluminal prosthesis of FIG. 3A surrounding an area where the first sleeve has been coupled to the endoluminal prosthesis through a heating process in accordance with some embodiments.

FIG. 3C illustrates a side-on view of the endoluminal prosthesis of FIG. 3A having the first sleeve disposed thereon following deployment thereof in accordance with some embodiments.

FIG. 4A is a cross-sectional view taken along line 4A-4A of FIG. 3B.

FIG. 4B is a cross-sectional view taken along line 4B-4B of FIG. 3B.

FIG. 4C is a cross-sectional view taken along line 4C-4C of FIG. 3B.

FIG. 5 illustrates a cross-sectional view of an alternative embodiment of an endoluminal prosthesis similar to that of FIG. 2A where the heating process was applied in a non-continuous manner around the circumference of the endoluminal prosthesis in accordance with some embodiments.

FIG. 6 illustrates a side-on view of a second embodiment the endoluminal prosthesis of FIG. 2A having a first sleeve disposed where the heating process was applied at a plurality of positions along the length of the endoluminal prosthesis thereon in accordance with some embodiments.

FIG. 7 is a flowchart illustrating a method of operations for securely coupling a first sleeve to an endoluminal prosthesis in accordance with some embodiments.

FIG. 8A illustrates a side-on view of the endoluminal prosthesis of FIG. 2A and a second sleeve to be disposed thereon in accordance with some embodiments.

FIG. 8B illustrates a side-on view of the endoluminal prosthesis of FIG. 2A and the second sleeve disposed thereon in accordance with some embodiments.

FIG. 8C illustrates a side-on view of the endoluminal prosthesis of FIG. 2A and the second sleeve disposed thereon following deployment thereof in accordance with some embodiments.

FIG. 9 illustrates an alternative embodiment of the second sleeve of FIGS. 8A-8C in accordance with some embodiments.

FIG. 10 is a flowchart of operations for a first method of forming a sleeve configured to restrain the diameter of an endoluminal prosthesis, such as a stent graft in accordance with some embodiments.

FIGS. 11A-11T are illustrations that depict the methodology of forming a sleeve configured to restrain the diameter of an endoluminal prosthesis as set forth in FIG. 10 in accordance with some embodiments.

FIG. 12 illustrates a stent graft including a framework, a graft, and a constrictive sleeve in accordance with some embodiments.

FIG. 13 illustrates a transverse cross-section of the stent graft of FIG. 12 in accordance with some embodiments.

FIG. 14 illustrates a detailed view of the stent graft of FIG. 12 in accordance with some embodiments.

FIG. 15 illustrates a longitudinal cross-section of the stent graft about a radiopaque marker band as indicated in FIG. 14 in accordance with some embodiments.

FIG. 16 illustrates the framework of the stent graft of FIG. 12 in accordance with some embodiments.

FIG. 17 illustrates a detailed view of the framework about a weld as indicated in FIG. 16 in accordance with some embodiments.

FIG. 18 illustrates a detailed view of the framework about another weld as indicated in FIG. 16 in accordance with some embodiments.

FIG. 19 illustrates a detailed view of the framework about another weld as indicated in FIG. 16 in accordance with some embodiments.

FIG. 20 illustrates a detailed view of the framework about a woven section as indicated in FIG. 16 in accordance with some embodiments.

FIG. 21 illustrates a distribution of radiopaque markers about a helical-section end portion of the framework in accordance with some embodiments.

FIG. 22 illustrates a distribution of the radiopaque markers about a woven-section end portion of the framework in accordance with some embodiments.

FIG. 23 illustrates a delivery system for delivering the endoluminal prosthesis i accordance with some embodiments.

FIG. 24 illustrates deploying the endoluminal prosthesis with the delivery system in accordance with some embodiments.

FIG. 25 illustrates a prior-art delivery system for delivering a prior-art endoluminal prosthesis.

FIG. 26 illustrates deploying the prior-art endoluminal prosthesis with the prior-art delivery system.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

In this description, references to “an embodiment,” “one embodiment” or the like, mean that the particular feature, function, structure or characteristic being described is included in at least one embodiment of the technique introduced herein. Occurrences of such phrases in this specification do not necessarily all refer to the same embodiment. On the other hand, the embodiments referred to also are not necessarily mutually exclusive.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. “Proximal” is used to indicate a portion, section, piece, element, or the like of a medical device or system intended to be near or relatively nearer to a clinician when the medical device or system is used on a patient. For example, “proximal” is used to indicate a portion, section, piece, element, or the like of the system near or relatively nearer to the clinician such as the control module while the clinician operates the control module with the one or more pads placed around the patient. A “proximal portion” or “proximal section” of the medical device or system includes a portion or section of the medical device or system intended to be near the clinician when the medical device is used on the patient. Likewise, a “proximal length” of the medical device or system includes a length of the medical device or system intended to be near the clinician when the medical device is used on the patient. A “proximal end” of the medical device or system is an end of the medical device or system intended to be near the clinician when the medical device or system is used on the patient. The proximal portion, the proximal section, or the proximal length of the medical device or system need not include the proximal end of the medical device or system. Indeed, the proximal portion, the proximal section, or the proximal length of the medical device or system can be short of the proximal end of the medical device or system. However, the proximal portion, the proximal section, or the proximal length of the medical device or system can include the proximal end of the medical device or system. Should context not suggest the proximal portion, the proximal section, or the proximal length of the medical device or system includes the proximal end of the medical device or system, or if it is deemed expedient in the following description, “proximal portion,” “proximal section,” or “proximal length” can be modified to indicate such a portion, section, or length includes an end portion, an end section, or an end length of the medical device or system for a “proximal end portion,” a “proximal end section,” or a “proximal end length” of the medical device or system, respectively.

“Distal” is used to indicate a portion, section, piece, element, or the like of a medical device or system intended to be near or relatively nearer a patient when the medical device or system is used on the patient. For example, “distal” is used to indicate a portion, section, piece, element, or the like of the system near or relatively nearer to the patient such as the one-or-more pads around the patient while a clinician operates the control module. A “distal portion” or “distal section” of the medical device or system includes a portion or section of the medical device or system intended to be near, relatively nearer, or even in the patient when the medical device or system is used on the patient. Likewise, a “distal length” of the medical device or system includes a length of the medical device or system intended to be near, relatively nearer, or even in the patient when the medical device or system is used on the patient. A “distal end” of the medical device or system is an end of the medical device or system intended to be near, relatively nearer, or even in the patient when the medical device or system is used on the patient. The distal portion, the distal section, or the distal length of the medical device or system need not include the distal end of the medical device or system. Indeed, the distal portion, the distal section, or the distal length of the medical device or system can be short of the distal end of the medical device or system. However, the distal portion, the distal section, or the distal length of the medical device or system can include the distal end of the medical device or system. Should context not suggest the distal portion, the distal section, or the distal length of the medical device or system includes the distal end of the medical device or system, or if it is deemed expedient in the following description, “distal portion,” “distal section,” or “distal length” can be modified to indicate such a portion, section, or length includes an end portion, an end section, or an end length of the medical device or system for a “distal end portion,” a “distal end section,” or a “distal end length” of the medical device or system, respectively.

Unless defined otherwise, all technical and scientific terms used herein have the same meaning as commonly understood by those of ordinary skill in the art.

### Endoluminal Prostheses

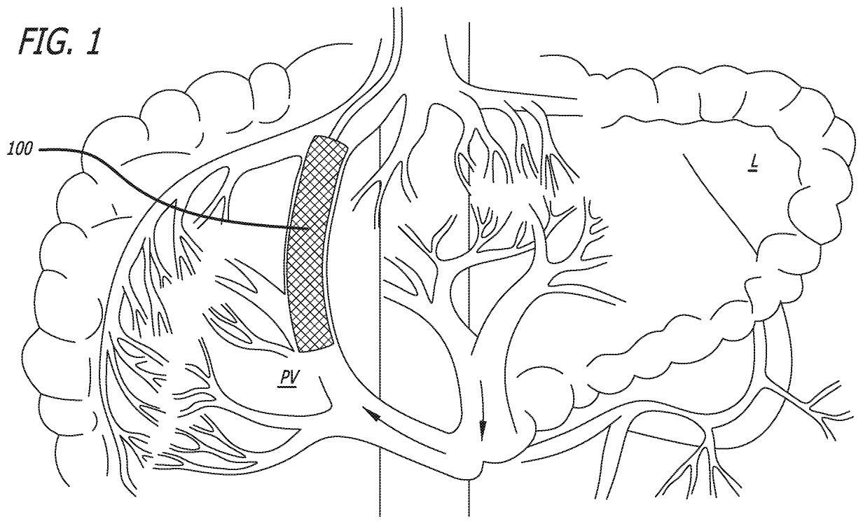

Referring now to FIG. 1, an endoluminal prosthesis in a portal vein in is shown in accordance with some embodiments. FIG. 1 illustrates an endoluminal prosthesis 200 or transjugular intrahepatic portosystemic shunt (“TIPS”) 200 in a portal vein PV carrying blood to a liver L in accordance with some embodiments. The endoluminal prosthesis 200, which can be placed in the portal vein PV by a clinician in a placement procedure with a percutaneous catheter delivery system, restores patency of the portal vein PV such that blood can easily flow through the liver rather than being blocked by blood flow-restricting blockages.

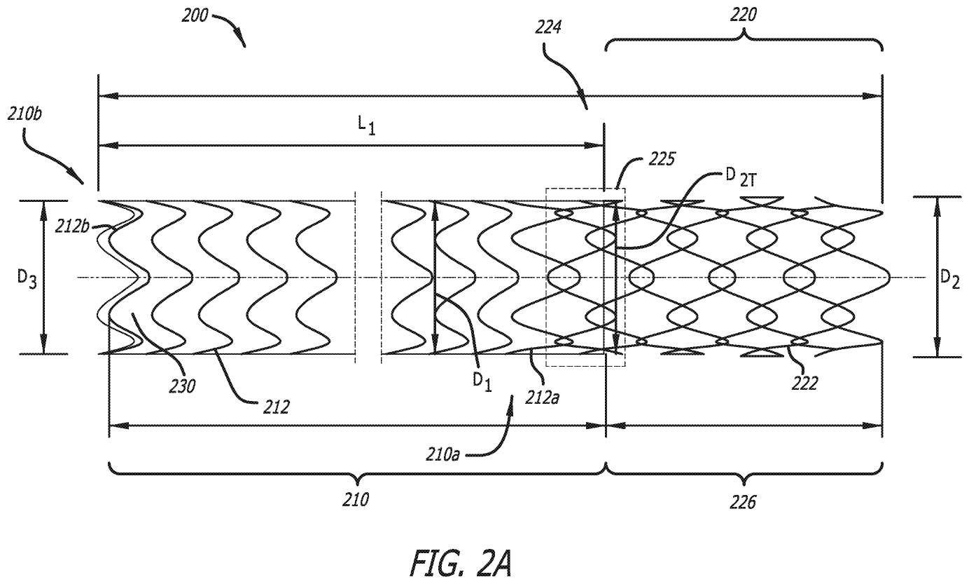

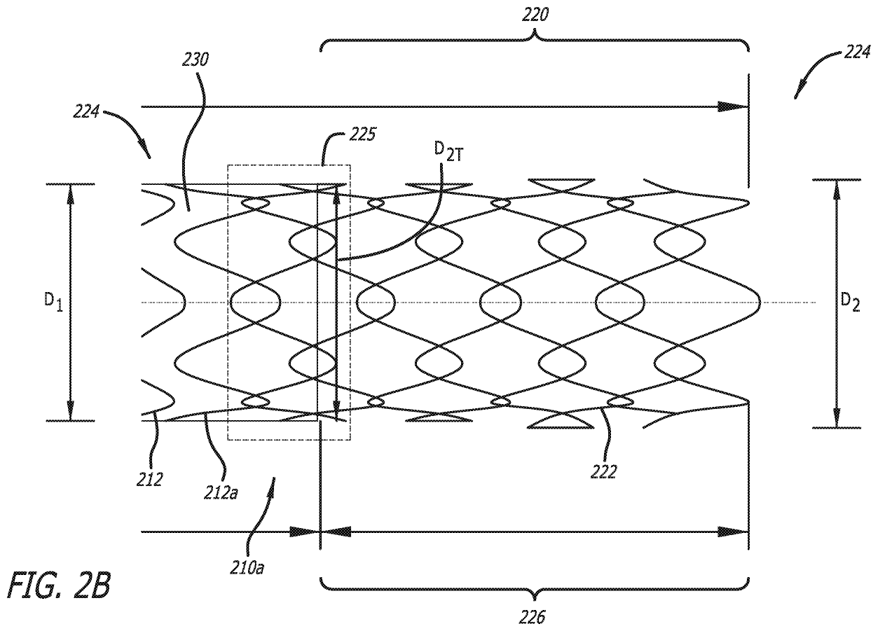

Referring to FIGS. 2A-2B, FIG. 2A illustrates a side-on view of an endoluminal prosthesis in accordance with some embodiments while FIG. 2B illustrates a detailed view of the endoluminal prosthesis of FIG. 2A about a coupling between a terminal frame and an annular member of a main frame of the endoluminal prosthesis in accordance with some embodiments. More particularly, FIG. 2A illustrates a side-on view of the endoluminal prosthesis 200 in accordance with some embodiments, while FIG. 2B illustrates a close-up view of the endoluminal prosthesis 200 about a woven coupling 225 between a terminal frame 220 and an annular member 212 of a main frame 210 of the endoluminal prosthesis 200.

As shown in FIGS. 2A-2B, the endoluminal prosthesis 200 includes a mixed frame of the main frame 210 and the terminal frame 220, as well as a tubular graft 230 over the main frame 210, each of which is described in further detail herein. While not shown in FIGS. 2A and 2B, the endoluminal prosthesis 200 includes an insertion state or compressed state for advancing the endoluminal prosthesis 200 through a patient's vasculature to the portal vein (PV). The endoluminal prosthesis 200 also includes an expanded state for placing the endoluminal prosthesis 200 in the portal vein. The endoluminal prosthesis 200 can be self-expanding in that it can expand, by itself, from the insertion state to the expanded state.

The main frame 210 includes or is formed of a number of annular members 212, for example, of nickel-titanium ally (nitinol) or polymers such as polyethylene terephthalate (PET) or polytetrafluoroethylene (PTFE) that are longitudinally spaced apart from each other. For example, a first-end annular member 212a is at a first end 210a of the main frame 210, while a second-end annular member 212b is at a second end 210b of the main frame 210. Each annular member 212 is formed of a strut 214 having a jagged or saw-tooth configuration that may vary with respect to their major dimension L1 and/or minor dimension L2. The longitudinal spacing of the annular members 212 in the main frame 210 is determined, in part, by the major dimension L1 or minor dimension L2 depending upon which dimension is longitudinal with the endoluminal prosthesis 200.

The terminal frame 220 includes or is formed of woven struts 122, for example, of nitinol. The terminal frame 220 includes a coupled end 224 and an uncoupled end portion 226 opposite the coupled end 224 that enables long-term placement of the endoluminal prosthesis 200 in the portal vein without shifting. The coupled end 224 of the terminal frame 220 is wovenly coupled to at least one of the first-end annular member 212a or the second-end annular member 212b in the woven coupling 225 respectively at the first end 210a or the second end 210b of the main frame 210. The woven coupling 225 is an extension of the woven struts 122 into the first-end annular member 212a or the second-end annular member 212b, which maintains flexibility in the endoluminal prosthesis 200 while providing collapse-preventing strength to the endoluminal prosthesis 200. In some embodiments, a second terminal frame 220 is present in the endoluminal prosthesis 200 (not shown), the second terminal frame 220 of the pair of terminal frames 220 is wovenly coupled to the second-end annular member 212b. Such a second terminal frame 220 can be the same as the first terminal frame 220 or different with respect to, for example, axial length or conicity. Regardless, having two terminal frames 220 without the tubular graft 230 may prevent “capping” of the portal vein when the endoluminal prosthesis 200 is placed therein. The uncoupled end portion 226 of the terminal frame 220 has a diameter D₂ that is greater than a diameter D₁ of the main frame 210, a diameter D_{2T} of the coupled end 224 of the terminal frame 220, and a diameter D₃ of the second end 210b in the insertion state or the expanded state of the endoluminal prosthesis 200.

The tubular graft 230 is over at least a majority of the main frame 210, under the majority of the main frame 210, or the majority of the main frame 210 is sandwiched between a pair of concentric tubular grafts 230. Any embodiment of the foregoing tubular graft 230 can extend from the first-end annular member 212a to the second-end annular member 212b such as up to the vertices of the struts 214, up to the woven coupling 225, or past the woven coupling 225 and up to a portion of the coupled end 224 of the terminal frame 220.

Any two adjacent annular members 212 are flexibly coupled together solely by a flexible coupling 215 provided by the tubular graft 230 between the two adjacent annular members 212 as shown in FIGS. 2A-2B. Such adjacent annular members 212 fixedly attached to the tubular graft 230 but are otherwise physically separate from each other or unconnected. A number of flexible couplings 215 between the annular members 212 imparts flexibility to the main frame 210 about the annular members 212. The flexible couplings 215 about the annular members 212 enable the endoluminal prosthesis 200 to keep a same length whether the endoluminal prosthesis 200 is in the insertion state or the expanded state. A relatively high degree of flexibility accommodates movement of surrounding tissue with little to no fatigue-based damage to the endoluminal prosthesis 200, little to no permanent deformation of the endoluminal prosthesis 200, or little to no change in cross-sectional area of the endoluminal prosthesis 200.

The tubular graft 230 can be a medically acceptable polymer such high-density polyethylene (“HDPE”) or expanded polytetrafluorethylene (“ePTFE”). Such a tubular graft 230 prevents tissue ingrowth about the main frame 210, thereby maintaining the flexibility of the main frame 210.

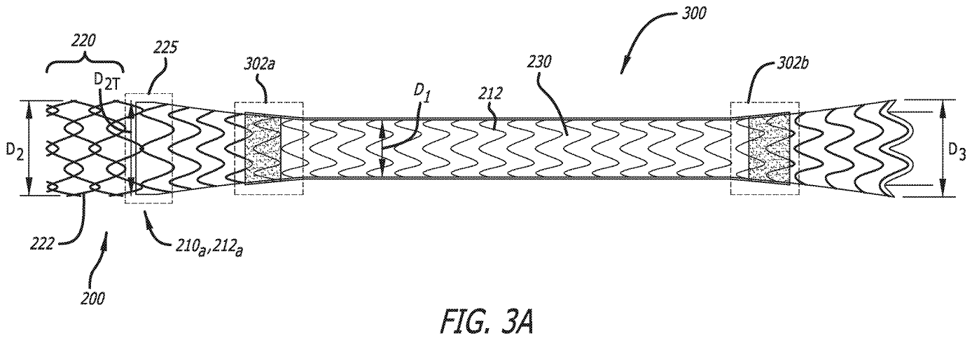

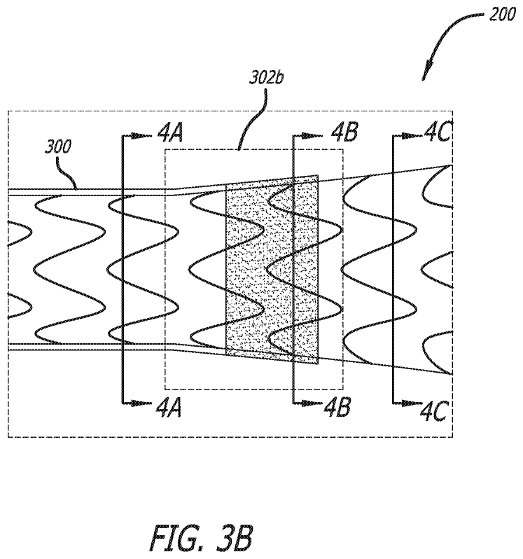

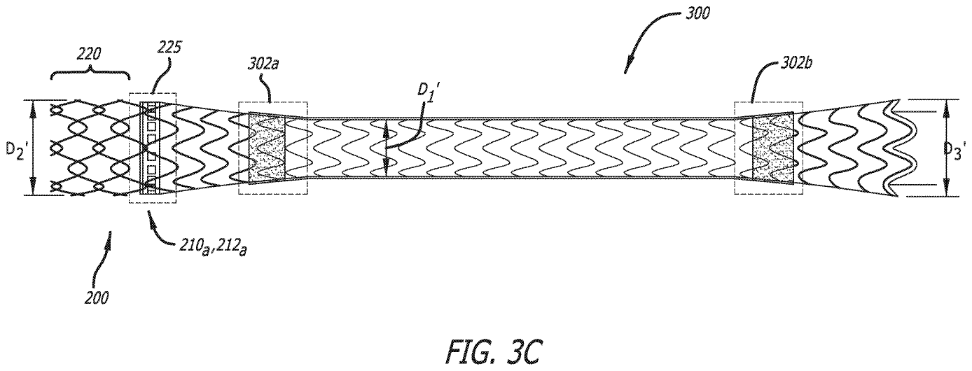

Referring now to FIG. 3A, a side-on view of the endoluminal prosthesis of FIG. 2A having a first sleeve disposed thereon is shown in accordance with some embodiments. FIG. 3A illustrates the endoluminal prosthesis 200 having a sleeve member 300 disposed around an exterior thereof and may be formed of HDPE or ePTFE. The sleeve member 300 provides a radial distensible feature in limiting the blood flow at a preset diameter of the stent graft 200. The sleeve member 300 has a length that is shorter than the length L₁ as shown in FIG. 2A, which, when the sleeve member 300 is disposed on the endoluminal prosthesis 200, results in a bone-shape appearance of the endoluminal prosthesis 200.

The resulting bone shape provides a transition slope from the cylindrical end of the sleeve member 300 to the endoluminal prosthesis 200, which results in axial sliding forces on the sleeve member 300 that attempt to shorten the sleeve member 300, when the sleeve member 300 is not fixedly coupled to the endoluminal prosthesis 200, e.g., when the sleeve member 300 is disposed on the endoluminal prosthesis 200 via interference fit, friction fit, etc. The axial sliding forces are especially present after expansion of the endoluminal prosthesis 200 and the sleeve member 300 post-deployment (e.g., within a patient vasculature). Additionally, and as a further result of the bone shape of the endoluminal prosthesis 200, local material of the endoluminal prosthesis 200 may accumulate at the transition slope during loading and crimping, which may lead to increased deployment forces. Additionally, the functional length of the endoluminal prosthesis 200 is compromised.

However, embodiments of the disclosure solve such problems through application of heat (heat tacking), resulting in a heat tacking of the ends of the sleeve member 300 to the endoluminal prosthesis 200. FIG. 3A illustrates that the sleeve member 300 may be heat tacked to the endoluminal prosthesis 200 at heat tacking areas 302a and 302b. Due to the heat tacking at the ends of the sleeve member 300, slippage does not occur along the length of the sleeve member 300 when mounted on the endoluminal prosthesis 200. Thus, the forces occurring at the transition slope do not alter the positioning of the sleeve member 300 and the disadvantages described above are solved. Further, during deployment, the change in the length of the sleeve member 300 is consistent with the change in the length of the endoluminal

### Prosthesis 200.

In some embodiments, the heat tacking includes placement of the sleeve member 300 around the exterior of the endoluminal prosthesis 200 and applying heat at the heat tacking areas 302a, 302b. In some embodiments, the heat may be within the temperature range of 180-200° C. at a pressure of approximately 20 bar (290 PSI). In other embodiments, the heat may be within the temperature range of 200-250° C. at a pressure less than 20 bar, and in one particular embodiment, the heat may be applied at 327° C. when material surfaces are in tight contact. In some embodiments, the heat may be applied for within the range of 2-10 seconds. In other embodiments, the heat may be applied for within the range of 3-5 seconds. Specifically, application of the heat results in the PTFE beginning to adhere to itself, i.e., the sleeve member 300 adhering to the endoluminal prosthesis 200. It should be understood that even when molten, PTFE does not flow due to its exceedingly high melt-viscosity. Thus, the specific form and dimensions of the sleeve member 300 adhering to the endoluminal prosthesis 200 will not be altered to the application of the heat at the heat tacking areas 302a, 302b.

In some embodiments, the sleeve member 300 may be formed of ePTFE and shaped as a tube as shown in FIG. 3A. The forming of the sleeve member 300 may include wrapping PTFE material around a tubular object such as a mandrel and applying compression and heat concurrently (at least partially overlapping in time). The result of the application of the compression and heat to the PTFE material wrapped around a mandrel is a thin sheath tube that may be configured in various lengths, thicknesses, and diameters. The radial expansion properties permitted by the resultant sleeve members are adjustable through adjustment of one or more of these parameters.

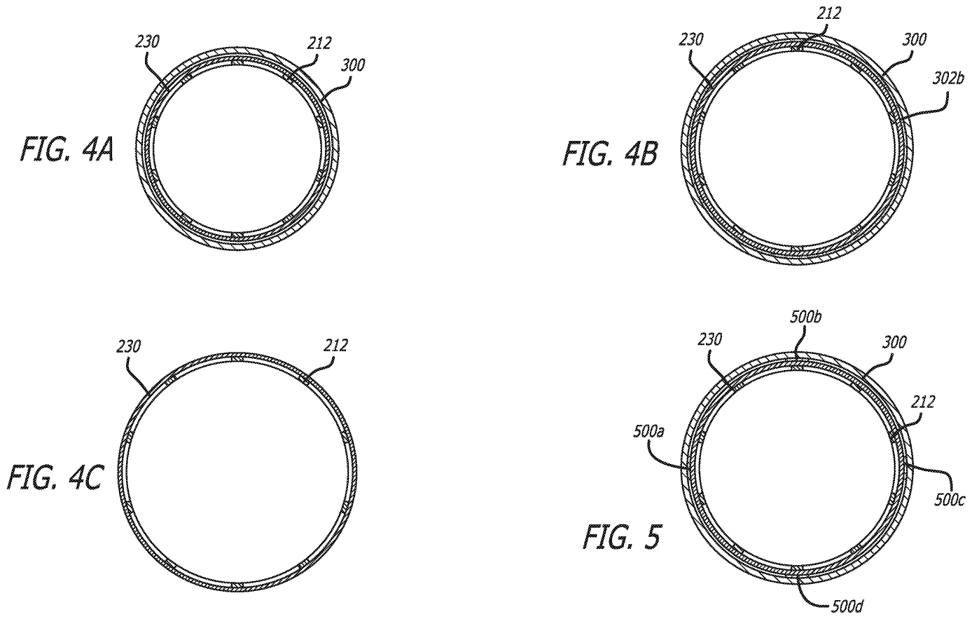

Referring to FIG. 3B, a detailed view of the endoluminal prosthesis of FIG. 3A surrounding an area where the first sleeve has been coupled to the endoluminal prosthesis through a combined pressure and heating process is shown in accordance with some embodiments. However, it should be understood that the first sleeve can be coupled to the endoluminal prosthesis via another process such as suturing in some other embodiments. Notwithstanding the foregoing, FIG. 3B illustrates a detailed view of the heat tacking area 302b and surrounding portions of the endoluminal prosthesis 200 and the sleeve member 300. In particular, FIG. 3B includes a first line 4A-4A indicating the position of the cross-sectional view of FIG. 4A illustrating the sleeve member 300 disposed on the endoluminal prosthesis 200 outside of the heat tacking area 302b. A second line 4B-4B is shown that indicates the position of the cross-sectional view of FIG. 4B illustrating the sleeve member 300 disposed on the endoluminal prosthesis 200 in the heat tacking area 302b. Additionally, FIG. 3B includes a third line 4C-4C indicating the position of the cross-sectional view of FIG. 4C illustrating a portion of the endoluminal prosthesis 200 that does not have the sleeve member 300 disposed

### Thereon.

Referring to FIG. 3C, a side-on view of the endoluminal prosthesis of FIG. 3A having the first sleeve disposed thereon following deployment thereof is shown in accordance with some embodiments. FIG. 3C illustrates that the heat tacking of the end portions of the sleeve member 300 prevented slippage during deployment. For example, both FIGS. 3A and 3C show that the sleeve member 300 begins five (5) struts 214 in from a second end 304b of the endoluminal prosthesis 200. Thus, while the distance between each strut 214 increases due to the deployment as shown in FIG. 3C, the positioning of the sleeve member 300 relative to the second end 304b of the endoluminal prosthesis 200 remained unchanged. The same applies to the heat tacking of the sleeve member 300 at the first end 304b of the endoluminal prosthesis 200 as can be seen in comparing the positioning of the sleeve member 200 relative to the first end 304b of the endoluminal prosthesis 200 in FIGS. 3A and 3C. FIG. 3C illustrates each of the diameters D₁′, D₂′, and D₃′ representing the diameters D₁, D₂, and D₃ following deployment.

Referring now to FIGS. 4A-4C, FIG. 4A provides a cross-sectional view taken along line 4A-4A of FIG. 3B, FIG. 4B provides a cross-sectional view taken along line 4B-4B of FIG. 3B, and FIG. 4C provides a cross-sectional view taken along line 4C-4C of FIG. 3B in accordance with some embodiments.

Referring now to FIG. 5, a cross-sectional view of an alternative embodiment of an endoluminal prosthesis like that of FIG. 2A where the heating process was applied in a non-continuous manner around the circumference of the endoluminal prosthesis is shown in accordance with some embodiments. In particular, the cross-sectional view of FIG. 5 provides an alternative embodiment to the illustration in FIG. 4B. The embodiment of FIG. 5 illustrates that the heat tacking need not be applied continuously around the endoluminal prosthesis 200 (as is shown in FIG. 4B) but may be applied at discrete areas around the endoluminal prosthesis 200 such as at locations 500a, 500b, 500c, and 500d. The four locations illustrated in FIG. 5 are representative and may be positioned differently. Further, heat tacking may occur at more or fewer locations.

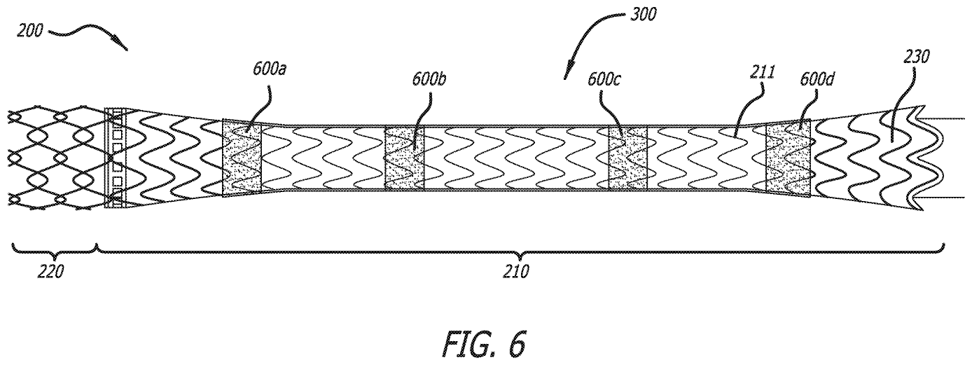

Referring to FIG. 6, a side-on view of a second embodiment the endoluminal prosthesis of FIG. 2A having a first sleeve disposed where the combined pressure and heating process was applied at a plurality of positions along the length of the endoluminal prosthesis thereon is shown in accordance with some embodiments. FIG. 6 illustrates an alternative embodiment to that shown in FIGS. 3A-3C by incorporating additional heat areas, namely, the heat tacking areas 600a, 600b, 600c, and 600d. FIG. 6 further illustrates that the alternative embodiment may utilize the same endoluminal prosthesis 200 and the sleeve member 300 as shown in FIGS. 3A-3C. Specifically, FIG. 6 illustrates an alternative to the heat tacking and not to the underlying structure of either of the endoluminal prosthesis 200 or the sleeve member 300.

Thus, as discussed above, the heat tacking includes placement of the sleeve member 300 around the exterior of the endoluminal prosthesis 200 and applying heat at the heat tacking areas 600a, 600b, 600c, and 600d. It should be understood that the illustration of FIG. 6 is illustrative and not in intended to be limiting. For example, an alternative number of heat tacking areas may be utilized. As with the description of FIG. 3A, in some embodiments, the heat applied while heat tacking may be within the temperature range of 180-200° C. at a first pressure. In other embodiments, the heat may be within the temperature range of 200-250° C. at a second pressure that is less than the first pressure, and in one particular embodiment, the heat may be applied at 327° C. when the material surfaces are in tight contact. Specifically, the heat tacking results in the PTFE beginning to adhere to itself, i.e., the sleeve member 300 adhering to the endoluminal prosthesis 200. It should be understood that even when molten, PTFE does not flow due to its exceedingly high melt-viscosity. Thus, the specific form and dimensions of the sleeve member 300 adhering to the endoluminal prosthesis 200 will not be altered to the application of the heat at the heat tacking areas 600a, 600b, 600c, and 600d.

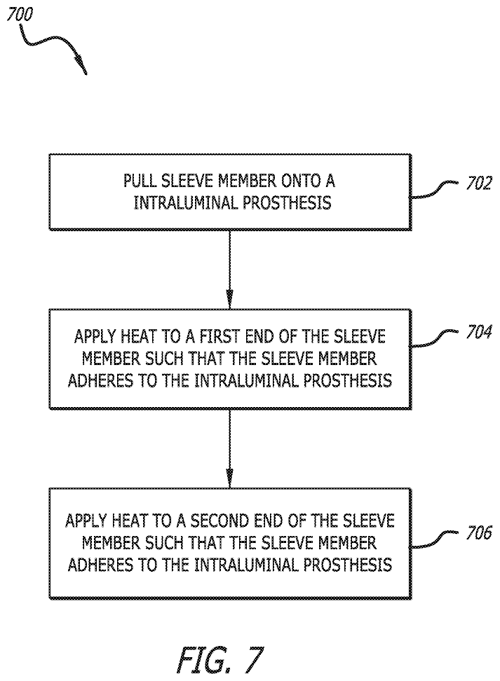

FIG. 7 is a flowchart illustrating a method of operations for securely coupling a first sleeve to an endoluminal prosthesis accordance with some embodiments. Each block illustrated in FIG. 7 represents an operation of the method 700. It should be understood that some operations of the method 700 may be optional and that additional operations may be included as separate operations or as sub-operations to those shown in FIG. 7. The method 700 begins with a sleeve member being pulled onto an endoluminal prosthesis that include an exterior tubular graft formed of ePTFE (block 702). The sleeve member may be formed by wrapping PTFE material around a tubular object such as a mandrel and applying compression and heat concurrently (at least partially overlapping in time). The result of the application of the compression and heat to the PTFE material wrapped around a mandrel is a thin sheath tube, i.e., the sleeve member.

The method 700 continues with the application of heat (heat tacking) at both ends of the sleeve member, resulting in a heat tacking of the ends of the sleeve member to the endoluminal prosthesis (blocks 707, 706). As discussed above, due to the heat tacking at the ends of the sleeve member, slippage does not occur along the length of the sleeve member when mounted on the endoluminal prosthesis, which is a problem in the current art and has been solved in embodiments of this disclosure.

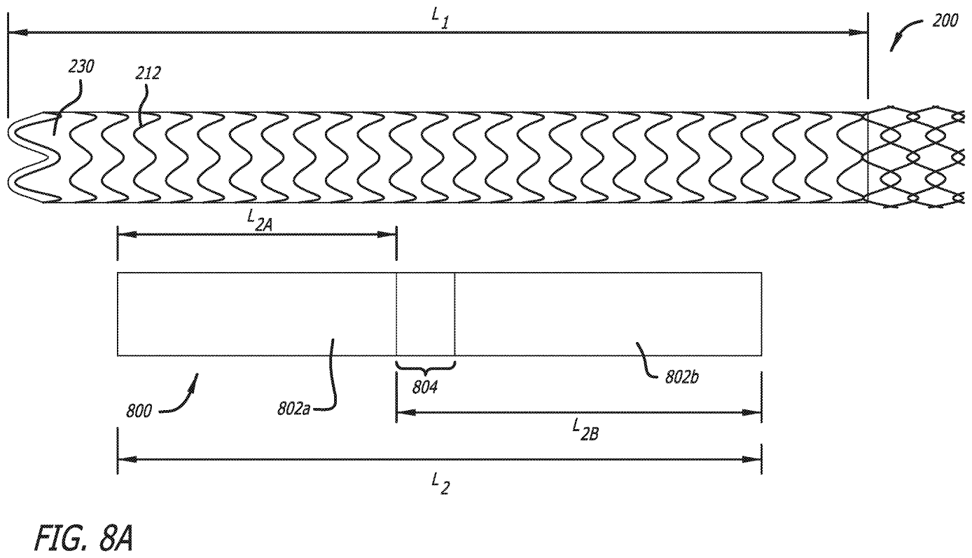

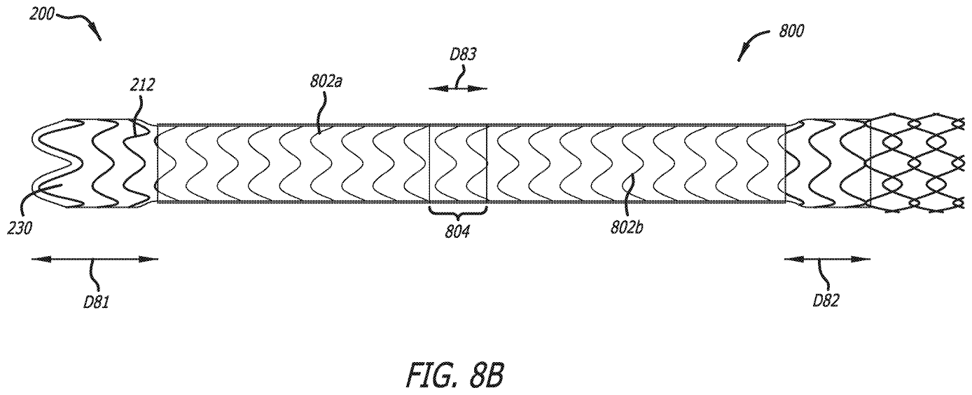

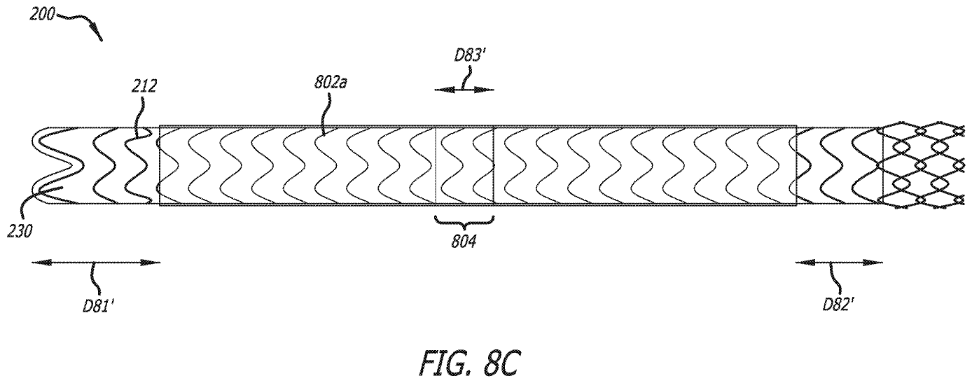

Referring now to FIG. 8A, a side-on view of the endoluminal prosthesis of FIG. 2A and a second sleeve to be disposed thereon is shown in accordance with some embodiments. The endoluminal prosthesis 200 is shown adjacent to a sleeve member 800. The endoluminal prosthesis 200 is shown to have a length L₁, which may be equivalent to L₁ of FIG. 2A, and the sleeve member 800 is shown to have an overall length L₂, which is comprised of sub-lengths L₂ₐ and L_{2b}. The sleeve member 800 is comprised of sub-restraint members 802a, 802b, which are two separate, independent tubular sleeves. Each sub-restraint member 802a, 802b may be formed by wrapping PTFE material around a tubular object such as a mandrel and applying compression and heat concurrently (at least partially overlapping in time) resulting in the thin sheath tubes shown. Additionally, while the sub-restraint members 802a and 802b are not securely coupled together, e.g., no heat tacking or other sintering, the sub-restraint members 802a, 802b form the sleeve member 800 when one sub-restraint member is placed inside the other forming an overlapping portion 804.

Referring to FIG. 8B, a side-on view of the endoluminal prosthesis of FIG. 2A and the second sleeve disposed thereon is shown in accordance with some embodiments. As is the case with the sleeve member 300 of FIG. 3A, the sleeve member 800 has a length L₂ that is shorter than the length L₁ of the endoluminal prosthesis 200. As a result, when the sleeve member 800 is disposed on the endoluminal prosthesis 200, the endoluminal prosthesis 200 takes on a bone-shaped appearance. FIG. 8B illustrates that outer portions of the endoluminal prosthesis 200 not covered by the sleeve member 800 span distances D₈₁ and D₈₂ and that the overlapping portion 804 spans the distance D₈₃ prior to deployment of the endoluminal prosthesis 200.

Referring now to FIG. 8C, a side-on view of the endoluminal prosthesis of FIG. 2A and the second sleeve disposed thereon following deployment thereof is shown in accordance with some embodiments. FIG. 8C illustrates the endoluminal prosthesis 200 having the sleeve member 800 disposed thereon following deployment. Specifically, FIG. 8C illustrates that the outer portions of the endoluminal prosthesis 200 not covered by the sleeve member 800 now span distances D₈₁′ and D₈₂′ and that the overlapping portion 804 spans the distance D₈₃′. Due to deployment (e.g., self-expansion of the endoluminal prosthesis 200), the distances D₈₁′ and D₈₂′ are greater than the distances D₈₁ and D₈₂ (FIG. 8B) while the distance D₈₃′ is less than the distance D₈₃ as the sub-restraint members 802a, 802b are configured to slide outwardly in opposite directions as the endoluminal prosthesis 200 expands. In contrast to FIGS. 3A-3C, the sub-restraint members 802a, 802b slide relative to the outer ends of the endoluminal prosthesis 200. However, the presence of the overlapping portion 804 having an initial distance D₈₃ enables the sub-restraint members 802a, 802b to shift positioning in an outward direction and lessen the overall shift of the sleeve member 800 relative to the outer ends of the endoluminal prosthesis 200.

While FIGS. 8A-8C do not illustrate that the outer ends of the sleeve member 800 are heat tacked to the endoluminal prosthesis 200, in some embodiments, the outer ends of the sleeve member 800 may be heat tacked to the endoluminal prosthesis 200 in a similar manner as described above with respect to at least FIG. 3A. Such embodiments in which the outer ends of the sleeve member 800 are heat tacked to the endoluminal prosthesis 200, the sub-restraint members 802a, 802b would not slide relative to the outer ends of the endoluminal prosthesis 200. Instead, expansion of the endoluminal prosthesis 200 during deployment would result in further adjustment of the width of the overlapping portion 804 (e.g., an additional decrease than that shown from D₈₃ to D₈₃′.

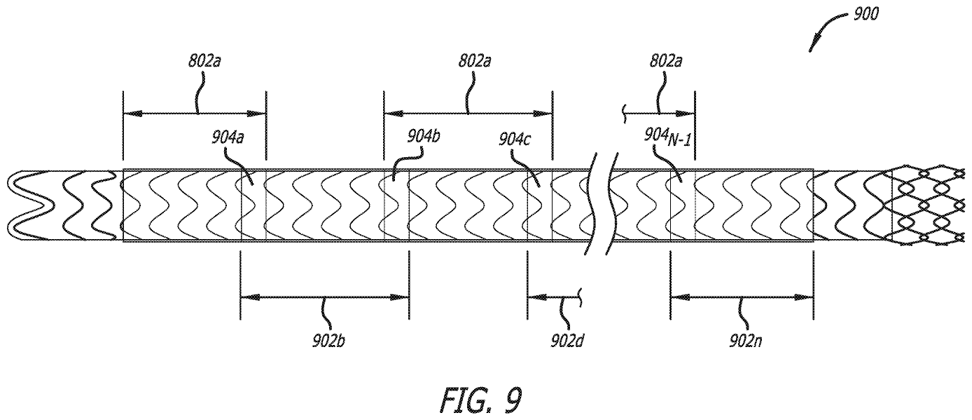

FIG. 9 illustrates an alternative embodiment of the second sleeve of FIGS. 8A-8C in accordance with some embodiments. FIG. 9 illustrates an alternative embodiment to that of FIGS. 8A-8C depicting a sleeve member 900 comprised of sub-restraint members 902a, 902b, 902c, 902d, 902_{N-1}, and 902_{N} (where N≥5 in this example), which are each separate, independent tubular sleeves. In other examples, an alternative number of sub-restraint members may be used, such as three or four.

Similar to the sleeve member 800, the sub-restraint members 902b, 902c, 902d, 902_{N-1}, and 902_{N} are not securely coupled together, e.g., no heat tacking or other sintering. The sub-restraint members 902a, 902b, 902c, 90d2, 902_{N-1}, and 902_{N} form the sleeve member 800 when one sub-restraint member is placed inside the other forming overlapping portions 904a, 904b, 904c, 904_{N-1}, etc. Each sub-restraint member 902a, 902b, 902c, 90d2, 902_{N-1}, and 902_{N} may be formed by wrapping PTFE material around a tubular object such as a mandrel and applying compression and heat concurrently (at least partially overlapping in time) resulting in the thin sheath tubes shown.

A third embodiment of constraining expansion of a transjugular intrahepatic portosystemic shunt (TIPS) is disclosed below. To supplement the disclosure of the use and purpose of a TIPS procedure set forth above, in a TIPS procedure, a shunt or channel is created between a hepatic vein and an intrahepatic branch of the portal vein. This may be done by passing a long, curved puncture needle from the hepatic vein through the liver parenchyma and into the portal vein. One example of a suitable access kit, and associated method of using the access kit to create a shunt between a hepatic vein and a portal vein is disclosed in PCT Application Publication No. WO 2022/242874, the disclosure of which is incorporated herein by reference in its entirety. After placement of a guidewire, the needle tract is dilated with a balloon dilation catheter and subsequently stented to maintain the shunt and resist the recoil of the liver parenchyma and tissue ingrowth. Interventionalists then measure the portosystemic pressure gradient (PSG), defined as the pressure difference between the portal vein and the right atrium. It has been established that reducing the PSG to less than 12 mmHg significantly reduces the risk of variceal rebleeding and formation of ascites.

In the event that the achieved pressure gradient is higher than 12 mmHg, the lumen of the stent graft may be expanded further by ballooning the stent graft and thus reducing the pressure gradient. In embodiments of the disclosure, to facilitate the tuning of the shunt diameter, the middle portion of a covered segment of a stent graft is constrained from the nominal inner diameter of 10 mm to an inner diameter of 6 mm or 8 mm. Dialing in a certain PSG may be suitable for an individual patient depending on the patient's symptoms, disease severity and individual risk of Hepatic Encephalopathy. In such cases, a clinician can apply a tailored expansion procedure (tailored to a patient's clinical need) to incrementally adjust the diameter of the stent graft, and thus the PSG. For example, after placement, the constrained middle segment may be post-dilated to the desired inner diameter using a balloon dilation catheter to achieve the desired PSG.

In one embodiment, a sleeve used to restrain the diameter of a stent graft may be formed of two different ePTFE materials through the application of pressure and heat. In some embodiments, the first material may be an ePTFE tubing having uniaxial strength in a first direction, high axial strength, and low radial strength. The second material may be an ePTFE membrane that is biaxially stretched having high radial and axial strength. In some embodiments, the first material is longitudinally expanded and the second material is biaxially expanded, which leads to different nodal distribution within the combined material following the application of pressure and heat discussed below. Specifically, longitudinal expansion leads to strong axial strength but low radial strength and biaxial expansion leads to strong axial and radial strength.

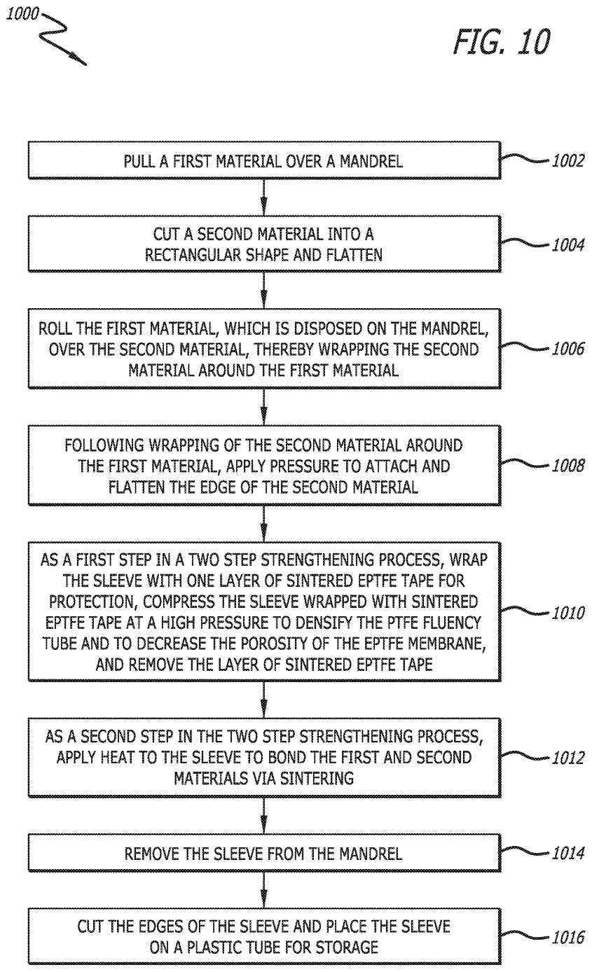

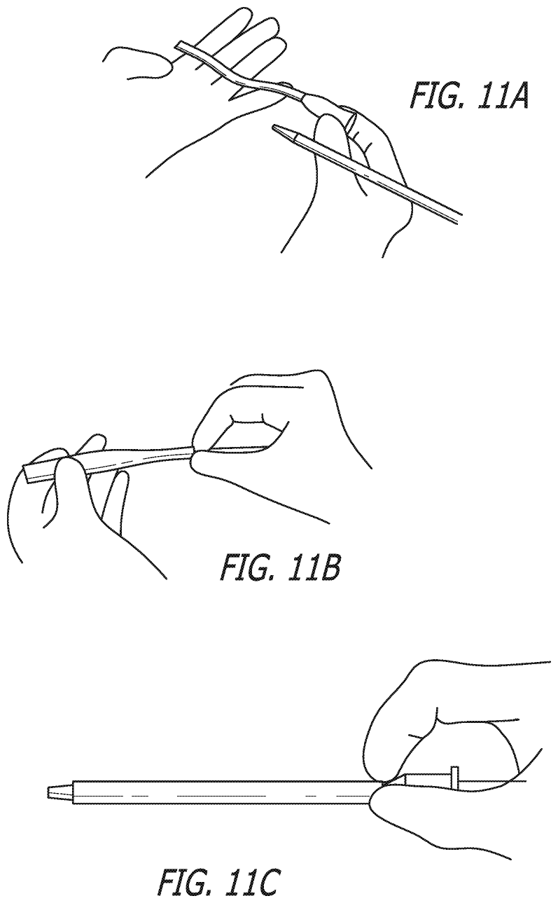

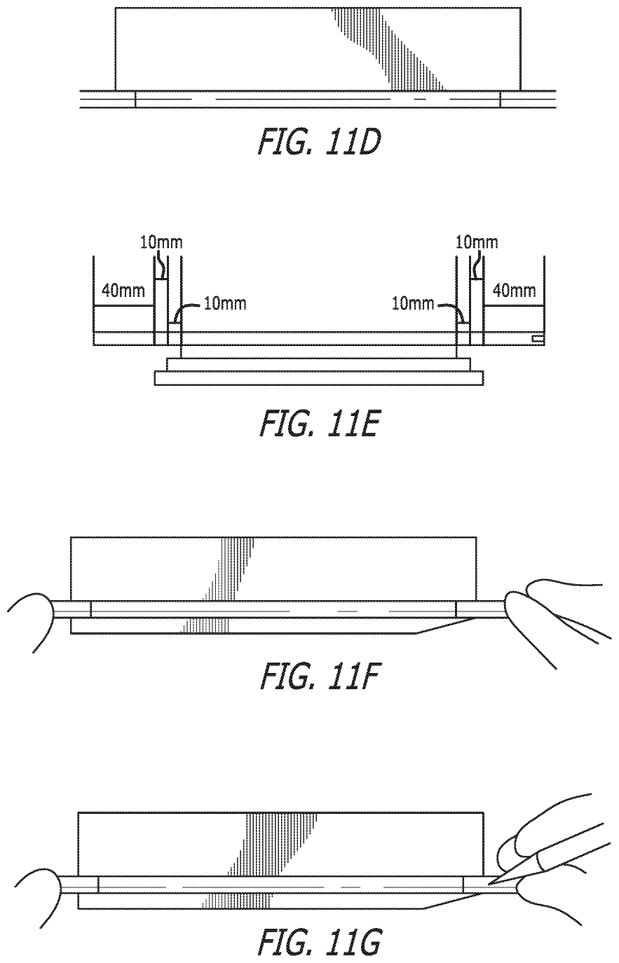

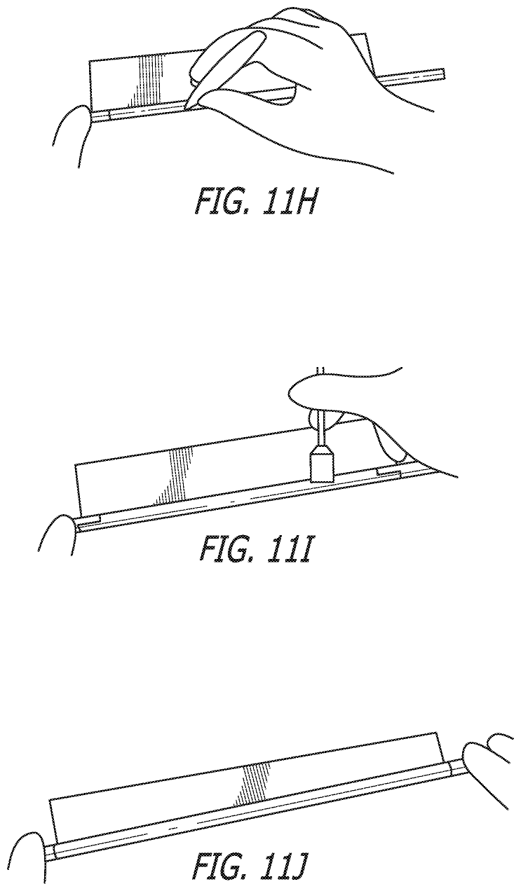

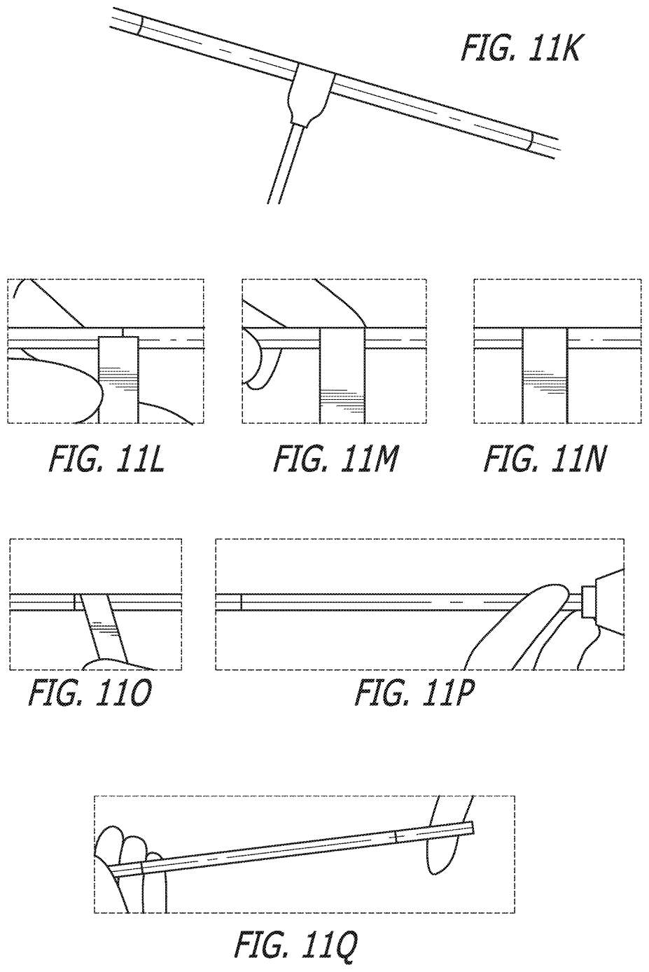

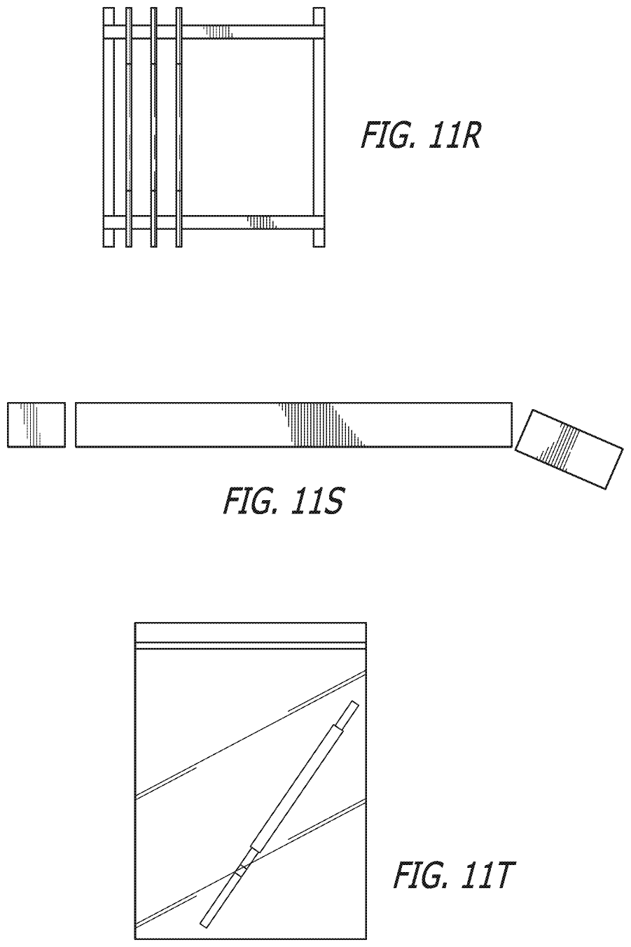

Referring now to FIG. 10, a flowchart of operations for a first method of forming a sleeve configured to restrain the diameter of an endoluminal prosthesis, such as a stent graft, is shown in accordance with some embodiments. Each block illustrated in FIG. 10 represents an operation of the method 1000, with various operations discussed in conjunction with FIGS. 11A-11T, which are illustrations that depict one embodiment of the method 1000. In a first operation, the first material is pulled over a 7.5 mm metallic mandrel (block 1002). For example, the first material may comprise an ePTFE fluid tube, which is pulled (loaded) onto the tapered end of the funnel as shown in FIG. 11A. The loaded funnel is then pulled onto the mandrel, which places the ePTFE fluid tube (first material) onto the mandrel, as shown in FIGS. 11B-11C. In a second operation, the second material is then cut into a rectangular shape and flattened (block 1004), which is shown in FIG. 11D, with an example diagram of measurements of the ePTFE fluid tube (first material), an ePTFE membrane (second material), and protective tape shown. The ePTFE membrane and protective tape are discussed below.

The first material, which is disposed on the mandrel, is then rolled over the second material, thereby wrapping the second material around the first material (block 1006). The operation (and sub-operations) of block 1006 are illustrated in FIGS. 11F-11J. The sub-operations forming the operation of block 1006 include placing an edge of the ePTFE membrane on the ePTFE fluid tube, e.g., with teasers, and attaching the edge of the ePTFE membrane to the ePTFE fluid tube by applying pressure with a cotton pad or swab. When the second material is wrapped around the first material, pressure is again applied with a cotton pad or swab to attach and flatten the edge of the second material (block 1008), which is illustrated in FIG. 11K. In this state, the first and second materials are in direct connection with each other but not bonded to each other and are radially too weak to withhold the chronical outward force (COF) of a self-expanding endoluminal prosthesis, e.g., a nitinol stent usually used for TIPS.

The combination of the first and second materials may be strengthened in a two-step process as follows. As a first step in the two-step process, the sleeve is wrapped with one layer of PTFE tape (e.g., sintered ePTFE tape) for protection, compressed at a high pressure (e.g., up to 250 bar) to densify the PTFE fluid tube and to decrease the porosity (distance between nods) of the ePTFE membrane, and the layer of PTFE tape is removed (block 1010).

The actions encompassing the first step are shown in at least FIGS. 11L-11P. In one embodiment, pressure of 250 bar was applied for two minutes. After the application of pressure and the removal of the layer of PTFE tape, the sleeve may have an opaque or slightly transparent look as compared to the white look before the application of pressure. The sleeve following the removal of the layer of PTFE tape is shown in FIG. 11Q.

As a second step in the two-step process, heat will be applied to the sleeve to bond the first and second materials (the ePTFE fluid tube and the ePTFE membrane) via “sintering” (block 1012). In some embodiments, the application of heat includes exposing the sleeve to a temperature of approximately 353° C. for 6-8 minutes. In some embodiments, the temperature may be 353° C. +/−3° C. In some embodiments, a plurality of mandrels having separate sleeves disposed thereon may be exposed to the high temperature at a single time. In such embodiments, the mandrels may be placed on a rack, which is placed in an oven at the applicable temperature. The rack including a plurality of mandrels is shown in FIG. 11R. The finished product is a strong compound of different ePTFE materials and can withhold force according to the material characteristics of the first and second materials, such as thickness, weight, number of layers etc. Additional operations in the method 1000 include removing the sleeve from the mandrel and cutting the edges of the sleeve, e.g., with a scalpel, and placing the sleeve on a plastic tube for storage (blocks 1014, 1016). The sleeve having cut edges is shown in FIG. 11S. The sleeve placed on the plastic tube may be stored, for example, in a plastic bag, as shown in FIG. 11T.

With the two-step process for strengthening the sleeve including the application of pressure and heat as discussed above, the sleeve achieves required properties such as tensile strength (elastic and plastic deformation), recoil behavior (the diameter decrease after dilation with a balloon) and diameter restriction. In a series of tests (tensile testing, stent diameter stability over time, etc.) at different temperatures, a variety of parameters (material, weight of material, sintering temperature and time, pressure and pressure time) were tested and the base weight of the membrane material. the sintering time, and time exposed to heat showed the biggest impact on the tensile strength and the recoil behavior of the sleeve.

It is noted that in some embodiments of the disclosure, a sleeve may be formed in the manner discussed above that restricts the diameter of an endoluminal prosthesis, e.g., nitinol stent, to 6 mm and which can be ballooned incrementally up to 10 mm. Such an embodiment may be advantageous for individuals that have a liver with smaller than average dimensions by starting with a smaller caliber shunt.

One illustrative example of a TIPS procedure includes the following steps. Like that set forth above, standard portal vein access techniques are first utilized to create a shunt and introduce a guidewire. Next, the parenchymal tract is dilated with a balloon dilation catheter of appropriate length and diameter. Further, a pressure measurement in the portal vein is completed and a pressure gradient is calculated. The length of the tract is then measured with a measuring catheter. Additionally, an appropriate stent graft length is selected to allow for full coverage of the parenchymal tract and the hepatic vein. With the assistance of fluoroscopy, a stiff guidewire is then advanced across the parenchymal tract into the portal vein. For example, the stiff guidewire may be an 0.035″ (0.89 mm) stiff guidewire.

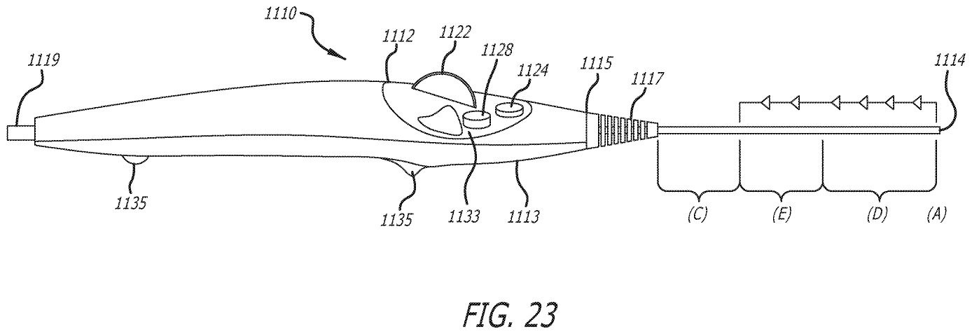

A box containing a stent graft within a sealed pouch may then be opened, and the pouch containing the stent graft is removed. The pouch should be carefully inspected to ensure that the sterile barrier has not been compromised. The pouch may be peeled and the tray containing the delivery system 1100 removed. Additionally, the delivery system 1100 is extracted from a tray. Further, the delivery system 1100 may be examined to ensure it has not been damaged during shipment and that its size, shape and condition are suitable for the procedure for which it is to be used. If it is suspected that the sterility or performance of the delivery system 1100 has been compromised, the delivery system 1100 should not be used. Notably, description of the delivery system 1100 is set forth below and shown in FIG. 23. Additional description is disclosed in PCT Application Publication No. WO 2022/083843, the disclosure of which is incorporated herein by reference in its entirety. For example, see FIG. 1 of PCT Application Publication No. WO 2022/083843.

The safety lock slider may then be verified as being in the locked position and that a push button is not depressed. The distal end of the delivery system 1100 should be visually inspected to ensure that the stent graft is contained within the sheath. The delivery system 1100 and the stent graft should not be utilized if the stent graft is partially deployed. The usable length portion of the delivery system 1100 should be wiped with gauze soaked with sterile saline. The delivery system 1100 guidewire lumen is to be flushed through the Luer port at the proximal end of the handle with sterile saline until saline drips from the distal tip of the delivery system 1100.

Subsequently, under radiographic guidance, the delivery system 1100 is advanced over the guidewire from the jugular vein access site through the hepatic vein and the liver tract into the portal vein until the radiopaque marker band located at the transition of the covered to the bare stent graft segment is positioned well inside the portal vein. Prior to stent graft deployment, the safety lock slider is moved to an unlock position. In one embodiment, the safety lock slider may be moved to the unlock position by pressing down and pulling the slider toward the proximal end of the handle from a locked position (distal position), where a closed lock icon may be visible, into the unlocked position (proximal position), where an open lock icon may be visible. The safety lock slider should be completely retracted to the proximal position, which is signified by the full visibility of a symbol for the unlocked position, e.g., an

### Open Lock Icon.

The stent graft release mechanism may then be activated by rotating the wheel on a topside of the handle proximally until a firm resistance to scrolling is felt. Confirm that the bare stent graft segment and a few millimeter portion of the covered stent graft segment with the radiopaque marker band are released. The delivery system 1100 may need to be pulled to the correct positioning with the expanded bare metal segment to the final position at the parenchymal/portal vein junction, which may be seen in FIG. 5a of PCT Application Publication No. WO 2022/083843. When the final stent graft position is confirmed, a button on the handle is pushed (depressed).

The wheel may continue to be rotated until a covered segment of the stent graft is fully deployed. Next, the delivery system 1100 is removed under fluoroscopy while maintaining guidewire access. The integrity of the delivery system 1100 should be visually confirmed at this point. Further, the stent graft segment inside the liver tract is post-dilated with a balloon dilation catheter as shown in FIG. 5b of PCT Application Publication No. WO 2022/083843. For the post-dilation, a balloon with a diameter sufficient to attain a targeted mmHg value should be utilized. The location and patency of the stent graft may then be verified using standard procedures as seen in FIG. 5c of PCT Application Publication No. WO 2022/083843. The guidewire and introducer sheath may be removed from the body and the entry site wound may be closed.

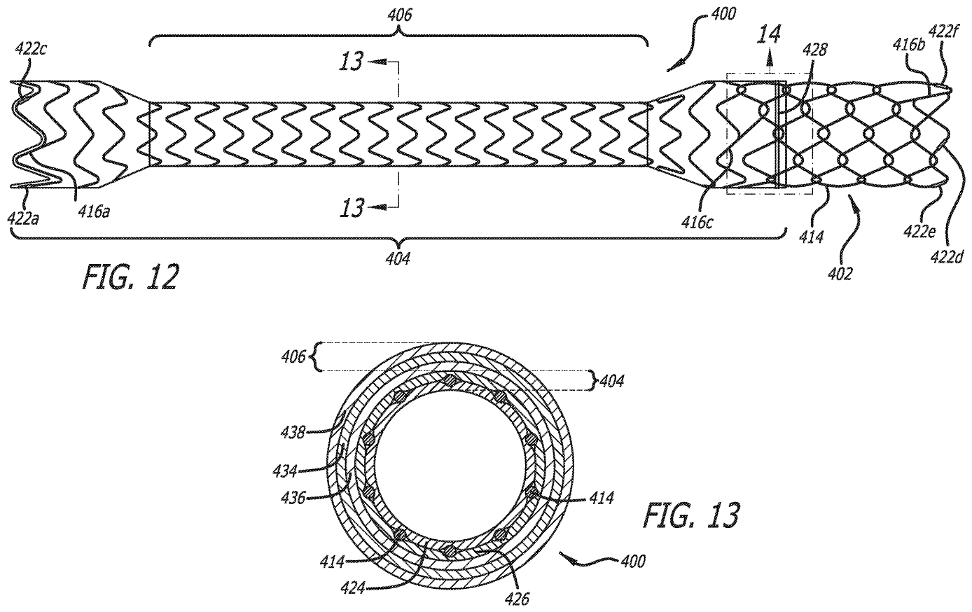

FIG. 12 illustrates a stent graft 400 in accordance with some embodiments. Notably, the stent graft 400 of FIG. 12 is in a ready-to-deploy or insertion state of the stent graft 400 with flared end portions of the stent graft 400. See any figure of FIGS. 1, 2A, 8A-8C, and 9 for an example of a deployed or expanded state of the stent graft 400 in which the stent graft 400 is expanded by a balloon.

As shown, the stent graft 400 includes a framework 402, a graft 404 encapsulating up to at least the helical section 408 of the framework 402, and a constrictive sleeve 406 over the outer graft member 426 of the graft 404. Each of the foregoing elements of the stent graft 400 are described in more detail below, beginning with the framework 402.

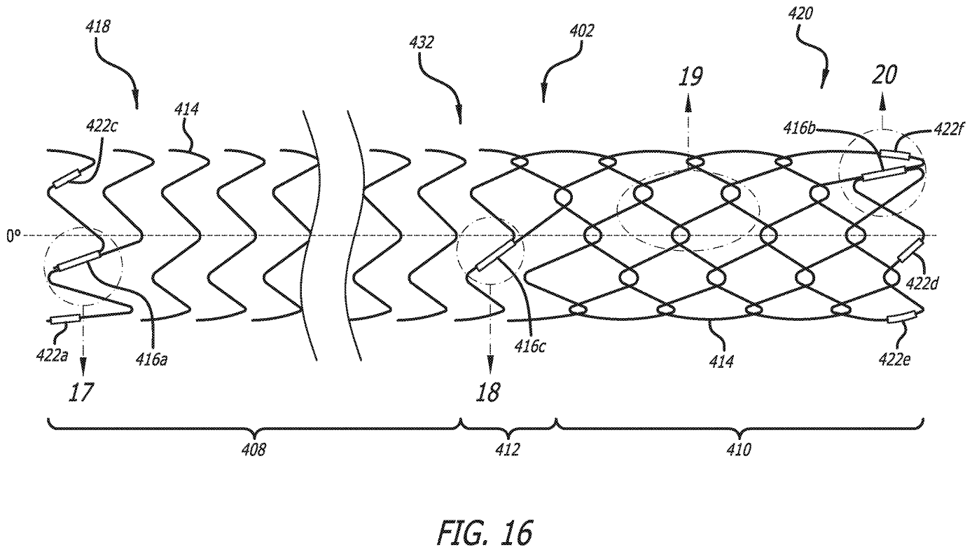

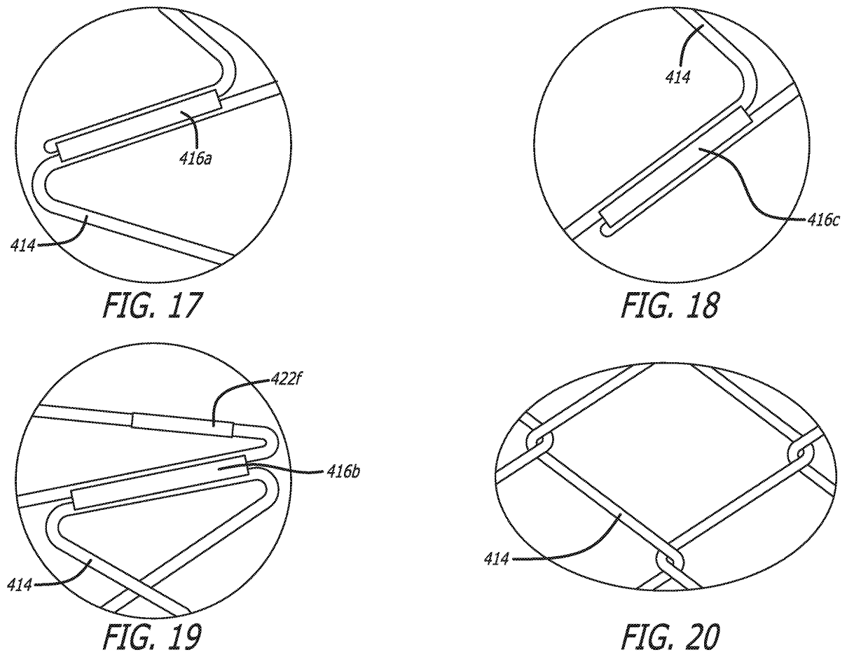

FIG. 16 illustrates the framework 402 of the stent graft 400 of FIG. 12 in accordance with some embodiments. FIGS. 17-20 illustrate detailed views of the framework 402 in accordance with some embodiments. Also, FIGS. 17-20 illustrate end portions of the framework 402 as if the single wire 414 forming the framework 402 was pulled out of the helical and woven sections 408 and 410 of the framework 402.

As shown, the framework 402 includes a helical section 408, a woven section 410, and a transitional or coupling section 412 between the helical section 408 and the woven section 410, various aspects of which can be gleaned from description of the mixed frame of the main frame 210 and the terminal frame 220 set forth above. Notably, the framework 402 of the stent graft 400 can consist of a single wire 414 (e.g., nitinol wire) and up to at least three wire welds 416. Indeed, the wire 414 can be formed into each section of the helical section 408, the woven section 410, and the transitional section 412 of the framework 402, and three wire welds 416 can be respectively distributed about the helical section 408, the woven section 410, and the transitional section 412 of the framework 402 to maintain a topology of the framework 402 whether the stent graft 400 is in the ready-to-deploy state or the expanded state thereof.

As shown, a first end portion of the wire 414 in a helical-section end portion 418 of the framework 402—or, stated differently, an end portion 418 of the helical section 408 opposite the transitional section 412—is welded with a weld 416 a to an internal portion of the wire 414 in the helical-section end portion 418 of the framework 402 or the foregoing end portion 418 of the helical section 408 of the framework 402. From the weld 416a, the wire 414 continues to wind around a central axis of the framework 402 in a counterclockwise direction away from the helical-section end portion 418 of the framework 402 when the framework 402 is viewed end-on from the helical-section end portion 418 of the framework 402. Indeed, the wire 414 continues to wind around the central axis of the framework 402 in the counterclockwise direction away from the helical-section end portion 418 of the framework 402, through the transitional section 412 of the framework 402, and to a woven-section end portion 420 of the framework 402—or, stated differently, an end portion 420 of the woven section 410 opposite the transitional section 412. In the woven-section end portion 420 of the framework 402 or the foregoing end portion 420 of the woven section 410, a looped portion of the wire 414 is welded with a weld 416b to an internal portion of the wire 414 in the end portion 420 of the woven section 410 of the framework 402. From the weld 416b, the wire 414 weaves into itself around the central axis of the framework 402 in a clockwise direction away from the woven-section end portion 420 of the framework 402 when the framework 402 is viewed end-on from the helical-section end portion 418 of the framework 402. Indeed, the wire 414 continues to weave around the central axis of the framework 402 in the clockwise direction away from the woven-section end portion 420 of the framework 402 to the transitional section 412 of the framework 402 where a second end portion of the wire 414 opposite the first end portion of the wire 414 is welded with a weld 416c to another internal portion of the wire 414. In accordance with the foregoing welds 416a, 416b, and 416c and the distribution thereof about the helical section 408, the woven section 410, and the transitional section 412 of the framework 402, a topology of the framework 402 is maintained whether the stent graft 400 is in the ready-to-deploy state or the expanded state.

Notably, the first end portion of the wire 414 in the helical-section end portion 418 of the framework 402 includes a rounded end of the wire 414. Likewise, the second end portion of the wire 414 in the transitional section 412 of the framework 402 includes a rounded end of the wire 414. Such rounded ends of the wire 414 obviate any potentially traumatic edges in the helical-section end portion 418 of the framework 402 and the transitional section 412 of the framework 402.

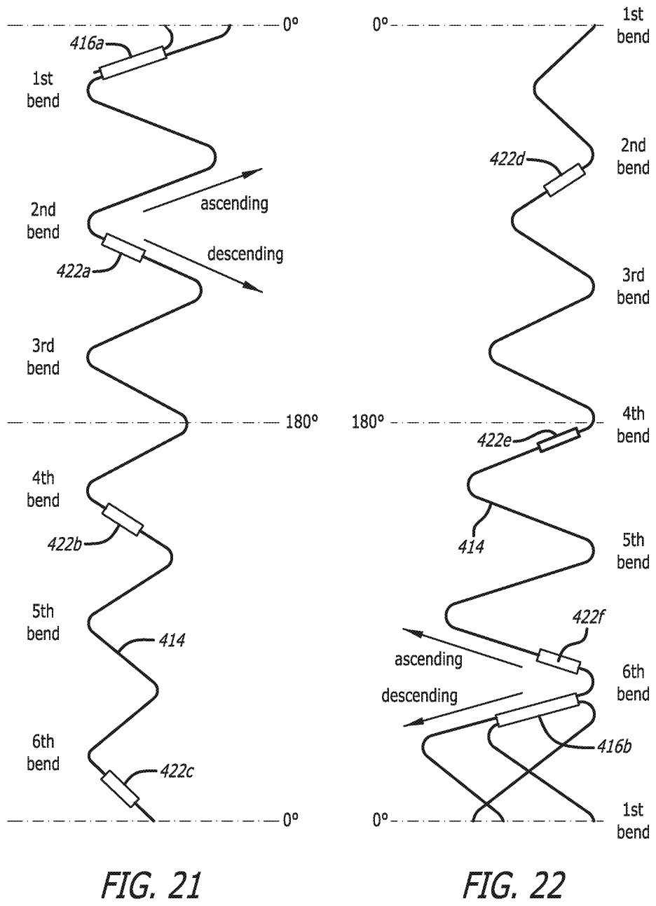

FIG. 16 illustrates a distribution of radiopaque markers 422a, 422b, 422c, ..., 422n, collectively, radiopaque markers 422, about the framework 402 in accordance with some embodiments. FIGS. 21 and 22 respectively illustrate the radiopaque markers 422 distributed about the helical-section end portion 418 and the woven-section end portion 420 of the framework 402 in accordance with some embodiments.

As shown in FIGS. 16 and 21, the radiopaque markers 422 can be distributed about the helical-section end portion 418 of the framework 402. Indeed, the end portion 418 of the helical section 408 opposite the transitional section 412 of the framework 402 can include up to at least three of the radiopaque markers 422 crimped onto the framework 402 approximately equidistant from each other around the end portion 418 of the helical section 408. Starting from the weld 416a and counting bends in the wire 414 opening away from the helical-section end portion 418 of the framework 402, the radiopaque markers 422 can be crimped onto the framework 402 about the second, fourth, and sixth bends in the wire 414 as exemplified by the radiopaque markers 422a, 422b, and 422c. Further, considering the legs of the bends that point away from their corresponding bends in the counterclockwise direction in which the wire 414 winds around the central axis of the framework 402 simply as “descending legs” to comport with the convention of FIG. 21, the radiopaque markers 422a, 422b, and 422c shown in FIG. 21 are crimped onto the framework 402 about the descending legs of the second, fourth, and sixth bends of the wire 414, respectively. So distributed, the radiopaque markers 422 about the helical-section end portion 418 of the framework 402 are easily observed by radiographic means no matter how the stent graft 400 is rotated about its central axis.

As shown in FIGS. 16 and 22, the radiopaque markers 422 can be additionally or alternatively distributed about the woven-section end portion 420 of the framework 402. Indeed, the end portion 420 of the woven section 410 opposite the transitional section 412 of the framework 402 can include up to at least three of the radiopaque markers 422 crimped onto the framework 402 approximately equidistant from each other around the end portion 420 of the woven section 410. Starting from the weld 416b and counting bends in the wire 414 opening away from the woven-section end portion 420 of the framework 402, the radiopaque markers 422 can be crimped onto the framework 402 about the second, fourth, and sixth bends in the wire 414 as exemplified by the radiopaque markers 422d, 422e, and 422f. Further, following the convention set forth above with respect FIG. 21 but noting that the descending legs of FIG. 22 point away from their corresponding bends in the clockwise direction in which the wire 414 weaves into itself around the central axis of the framework 402, the radiopaque markers 422d and 422e shown in FIG. 22 are crimped onto the framework 402 about the descending legs of the second and fourth bends of the wire 414, respectively. Notably, due to the presence of the weld 416b welding the descending leg of the sixth bend of the wire 414 to the looped end of the wire 414, the radiopaque marker 422f shown in FIG. 22 is crimped onto the framework 402 about the ascending leg of the sixth bend of the wire 414, which ascending leg, like all ascending legs shown in FIGS. 21 and 22, is opposite its corresponding descending leg. So distributed, the radiopaque markers 422 about the woven-section end portion 420 of the framework 402 are easily observed by radiographic means no matter how the stent graft 400 is rotated about its central axis.

Notably, the radiopaque markers 422 can consist essentially of tantalum, tungsten, or some other radiopaque metal or alloy.

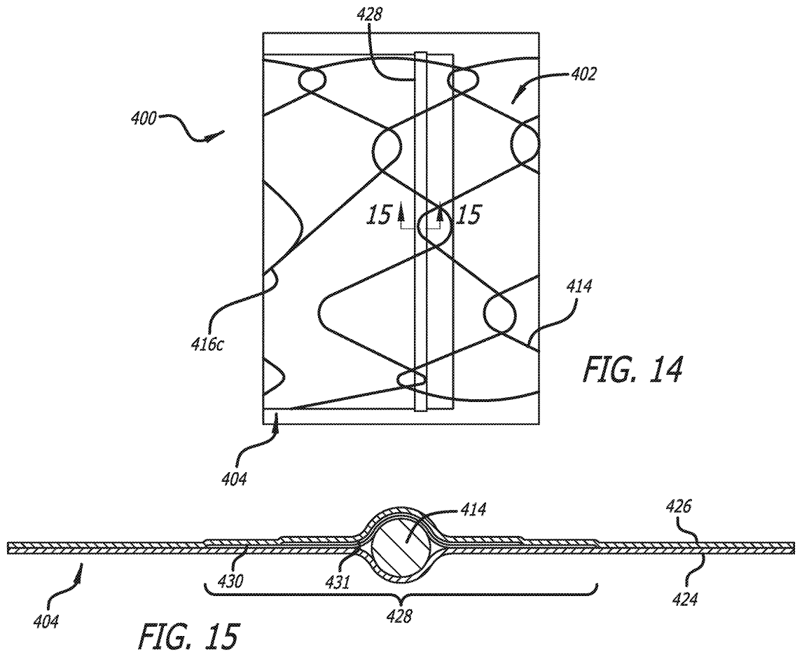

FIG. 12 illustrates the stent graft 400 and its graft 404 in accordance with some embodiments, and FIG. 14 illustrates a detailed view thereof. FIG. 13 illustrates a transverse cross-section of the stent graft 400 and its graft 404 in accordance with some embodiments.

As shown, the graft 404 includes an inner graft member 424 and an outer graft member 426 encapsulating up to at least the helical section 408 of the framework 402, various aspects of which can be gleaned from description of the tubular graft 230 set forth above, particularly the pair of concentric tubular grafts 230 that sandwich the main frame 210. Notably, each graft member of the inner graft member 424 and the outer graft member 426 can follow the bends in the wire 414 as shown in FIG. 12, thereby providing a scalloped, fluted, or waved edge in at least the helical-section end portion 418 of the framework 402, which eliminates unsupported portions of the graft 404 that could otherwise occlude a biological vessel such as the hepatic vein or the portal vein when shunted by the stent graft 400. Further, each graft member of the inner graft member 424 and the outer graft member 426 can independently consist essentially of ePTFE, optionally, impregnated with carbon for reduced thrombogenicity. Indeed, in some embodiments, the inner graft member 424 consists essentially of ePTFE impregnated with carbon while the outer graft member 426 consists essentially of ePTFE without carbon impregnated therein.

FIG. 14 illustrates a detailed view of the stent graft 400 and a radiopaque marker band 428 thereof in accordance with some embodiments. FIG. 15 illustrates a longitudinal cross-section of the stent graft 400 about the radiopaque marker band 428 as indicated in FIG. 14 in accordance with some embodiments.

As shown, the stent graft 400 can include the radiopaque marker band 428 around the transitional section 412 of the framework 402, various aspects of which can be gleaned from description of the radiopaque marker band located at the transition of the covered to the bare stent graft 400 segment set forth above. Notably, the radiopaque marker band 428 can consist essentially of tantalum, tungsten, or some other radiopaque metal or alloy printed on a ring member 430 and disposed between the framework 402 and the outer graft member 426 of the graft 404. Indeed, as shown in FIGS. 3C, 6, and 15, the foregoing radiopaque metal or alloy can be printed as discrete quadrilaterals 431 (e.g., squares) on the ring member 430 and disposed between the framework 402 and the outer graft member 426 of the graft 404. In addition to that set forth above for the radiopaque marker band, the radiopaque marker band 428 advantageously facilitates confirmation of the orientation of the stent graft 400 when the stent graft 400 is placed in a biological vessel such as the hepatic vein or the portal vein. Indeed, even in shorter versions of the stent graft 400, the radiopaque marker band 428 is closer to the woven-section end portion 420 of the framework 402, thereby facilitating radiographic differentiation between the helical-and woven-section end portions 418 and 420 of the framework 402 for confirming the orientation of the stent graft 400 in the biological vessel.

FIG. 12 illustrates the stent graft 400 and its constrictive sleeve 406 in accordance with some embodiments. FIG. 13 illustrates a transverse cross-section of the stent graft 400 and its constrictive sleeve 406 in accordance with some embodiments.

As shown, the constrictive sleeve 406 is over the outer graft member 426 of the graft 404 akin to that set forth above for the sleeve member 300, description for which can be referenced for the constrictive sleeve 406 when suitable. Indeed, both the constrictive sleeve 406 and the sleeve member 300 can have a length shorter than that of the graft 404. Further, both the constrictive sleeve 406 and the sleeve member 300 can be approximately centered over the graft 404. Accordingly, both the end portion 418 of the helical section 408 opposite the transitional section 412 and an end portion 432 of the helical section 408 proximate the transitional section 412 extending through the end portion 420 of the woven section 410 opposite the transitional section 412 can be flared in the ready-to-deploy state of the stent graft 400, thereby providing the bone-shaped appearance of the stent graft 400 or the endoluminal prosthesis 200. However, it should be understood that the constrictive sleeve 406 and the sleeve member 300 can vary substantially from each other as well. In an example, while both the constrictive sleeve 406 and the sleeve member 300 can consist essentially of ePTFE, the constrictive sleeve 406 can include a tube 434 consisting essentially of ePTFE that is disposed between an inner membrane 436 and an outer membrane 438 consisting essentially of ePTFE, wherein the inner membrane 436, the outer membrane 438, or both the inner membrane 436 and the outer membrane 438 have a greater resistance to radial expansion than the tube 434. In another example, the tube 434 serves as a structural base of the constrictive sleeve 406 with the inner membrane 436 over the tube 434 and the outer membrane 438 over the inner membrane 436. In another example, the tube 434 continues to serve as the structural base of the constrictive sleeve 406 but the inner membrane 436 and the outer membrane 438 correspond to an inner layer and an outer layer of a single, continuous membrane consisting essentially of ePTFE wrapped twice around the tube 434. Notably, such embodiments of the constrictive sleeve 406 can be sintered to fuse ePTFE interfaces together for an integrated structure that better resists expansion. While the sleeve member 300 is fixedly coupled to the tubular graft 230 via heat tacking or the like as set forth above, the constrictive sleeve 406 need not be heat tacked or the like to the graft 404. In some embodiments, for example, the constrictive sleeve 406 is coupled to the graft 404 exclusively by an interference fit. Such a constrictive sleeve 406 is configured to resist radial expansion of the stent graft 400 over a length of the stent graft 400 commensurate with the constrictive sleeve 406 until radially expanded with an elongate balloon in vivo as shown in FIG. 1.

Notably, the inner graft member 424 can have a wall thickness of 40-120 μm, including 60-100 μm, such as 70-90 μm, for example, 75-85 μm. For example, the inner graft member 424 can have a wall thickness of about 80 μm. The outer graft member 426 can have a wall thickness of 100-200 μm, including 120-180 μm, such as 140-160 μm, for example, 145-155 μm. For example, outer graft member 426 can have a wall thickness of about 150 μm. Taking the foregoing into consideration, the graft 404 (without consideration of the constrictive sleeve 406 thereover) can have a wall thickness of 250-400 μm, including 300-380 μm, such as 320-370 μm, for example, 330-360 μm, wherein the diameter of the graft 404 is 8-12 mm, such as about 10.5 mm. For example, the graft 404 can have a wall thickness of about 350 μm. Lastly, with respect to the graft 404, the inner graft member 424 and the outer graft member 426 thereof can extend about 40-50 mm over the over the helical section 408 and the connection of the helical section 408 to the woven section 410. About 20 mm of the woven section 410 can be left uncovered on the distal end for placement in the portal vein.

The constrictive sleeve 406 can be made by positioning an extruded ePTFE unsintered tube corresponding to the tube 434 over a mandrel, wrapping one or more ePTFE unsintered membranes around the ePTFE unsintered tube to form a corresponding number of layers of a layered structure, applying radial pressure to the layered structure, and sintering the layered structure in an oven at, for example, 355° C. for 8 minutes to form the integrated structure of the constrictive sleeve 406. Advantageously, the one or more ePTFE unsintered membranes are stronger than the ePTFE unsintered tube and better resists expansion. If one ePTFE unsintered membrane is wrapped twice around the ePTFE tube, the resulting constrictive sleeve 406 includes the tube 434 as the structural base and the inner layer and the outer layer of the ePTFE membrane thereover, wherein the inner layer and the outer layer of the ePTFE unsintered membrane is akin to a single, continuous membrane of the inner membrane 436 and the outer membrane 438. If two ePTFE unsintered membranes are wrapped around the ePTFE tube, the resulting constrictive sleeve 406 includes the tube 434 as the structural base and the inner membrane 436 and the outer membrane 438 thereover. In one example, an ePTFE unsintered membrane sheet is wrapped twice around the ePTFE unsintered tube such that there is a first membrane layer, a second membrane layer, and an overlap of about 1-3 mm. It should be appreciated that additional wrapped layers are also within the scope of the invention, whether from a single unsintered membrane sheet of ePTFE, separate unsintered membrane sheets of ePTFE, or separate unsintered ePTFE membranes formed into tubular structures. For example, an ePTFE unsintered membrane sheet could be wrapped around a base ePTFE tube three, four, five times, etc. depending on the desired strength of the constrictive sleeve. After the constrictive sleeve 406 is made, it can be positioned over the outside of the graft 404 and the proximal and distal ends of the stent graft 400 can be flared using a balloon.

### Delivery System

FIG. 23 shows a delivery system 1110 for deploying the endoluminal prosthesis 100 in accordance with some embodiments. As shown, a handle 1112 includes a first component 1113 and a second component 1115 arranged on top of each other and connected to one another. At one end of the handle 1112, a flexible tip 1117 is provided. At the end of the flexible tip 1117, a catheter 1114 that is suitable for insertion into a patient's vasculature is arranged. This catheter 1114 can be advanced through a patient's vasculature and provide, at the distal end thereof, the endoluminal prosthesis 100. The catheter 1114 has provided thereon a prosthesis-holding portion (not shown) at the distal end thereof as well as a prosthesis-constraining sheath (not shown) that covers the prosthesis-holding portion.

The handle 1112 has provided on its upper surface a recess 1133 that is arranged so that a surgeon can rest his or her thumb thereon. At the opposite side of the handle 1112 relative to the recess 1133, two protrusions 1135 are arranged so as to help the surgeon in holding the delivery system 1110 without it slipping. A surgeon can grip the handle 1112 such that his or her thumb rests on the recess 1133 while the remaining fingers are arranged between the protrusions 1135, thereby securing the delivery system 1110 by the handle 1112. The protrusions 1135 are provided at two longitudinal spaced apart positions of the handle 1112.

A thumbwheel 1122 on the handle 1112 sticks out of an opening of the handle 1112 that is provided inside the recess 1133. The thumbwheel 1122 has a structured surface that increases the friction thereof so as to make operating it easier. The thumbwheel 1122 is coupled to a spindle (not shown) that is, in turn, coupled to a pull tab (not shown). This pull tab is coupled to the prosthesis-constraining sheath so that proximal movement of the pull tab pulls the prosthesis-constraining sheath proximally to uncover the prosthesis-holding portion and, thus, deploy the endoluminal prosthesis 100 that is held at the prosthesis-holding portion. By operating the thumbwheel 1122, the pull tab winds onto the spindle so that the pull tab is pulled proximally (to the left hand side in FIG. 23).

In FIG. 23, a first release mechanism 1128 and a second release mechanism 1124 are provided. The first release mechanism 1128 is actuated by means of a button whilst the second release mechanism 1124 is operable by means of a slider. When the second release mechanism 1124 has not been operated, a structure blocks withdrawal of the prosthesis-constraining sheath so that the prosthesis-holding portion cannot be even partially uncovered. That is, unless the second release mechanism 1124 has been actuated, the endoluminal prosthesis 100 provided at the prosthesis-holding portion cannot even be partially deployed. However, when sliding the slider of the second release mechanism 1124 to the actuated position, this blocking of the proximal withdrawal of the pull tab is relieved so that rotation of the thumbwheel 1122 can pull the pull tab proximally. This allows for a partial deployment of the endoluminal prosthesis 100 that is arranged at the prosthesis-holding portion at the distal end of the catheter 1114.

When the pull tab is pulled further proximally by means of operating the thumbwheel 1122, the proximal pulling of the pull tab is blocked by engagement with a first interference feature. Such engagement can also be selectively released by actuating the first release mechanism 1128 by means of pushing the associated button. This releasable blocking of further withdrawal of the pull tab can correspond to a partial deployment of the endoluminal prosthesis 100 arranged at the distal end of the catheter 1114. This can correspond to the selective and partial release of the uncovered portion of the endoluminal prosthesis 100. As pointed out before, this is highly advantageous when it comes to deploying and placing the endoluminal prosthesis 100 in a human liver. Once the uncovered portion has been deployed, the surgeon can actuate the first release mechanism 1128 to release the blocking of further withdrawal of the pull tab to thus completely release the endoluminal prosthesis 100.

In the structure shown in FIG. 23, different positions and portions have been marked up on the catheter 1114. Position A denotes the position up to which the prosthesis-constraining sheath extends when the prosthesis-holding portion is fully covered. This position corresponds to the position where the further withdrawal of the pull tab is blocked by means of the second release mechanism 1124 not being actuated. When the second release mechanism 1124 is actuated, the prosthesis-constraining sheath can be pulled back over a distance denoted by the letter ‘D.’ This partial withdrawal corresponds to only the uncovered portion of the endoluminal prosthesis 100 being exposed to the patient's body.

Portion E corresponds to a sufficiently full withdrawal of the prosthesis-constraining sheath such that the entirety of the prosthesis-holding portion (and thus of the endoluminal prosthesis 100) is uncovered so that the endoluminal prosthesis 100 can be deployed. This would correspond to the full deployment configuration.

Portion C is that portion of the catheter 1114 that is not used for carrying the endoluminal prosthesis 100. That is, this portion serves to provide enough of a length of the catheter 1114 so as to reach the deployment position of the endoluminal prosthesis 100.

Notably, the catheter 1114 can have provided therein a lumen suitable for leading a guidewire therethrough. This guidewire can extend, at the proximal end of the handle 1112, through a connector 1119 that can be a Luer connector.

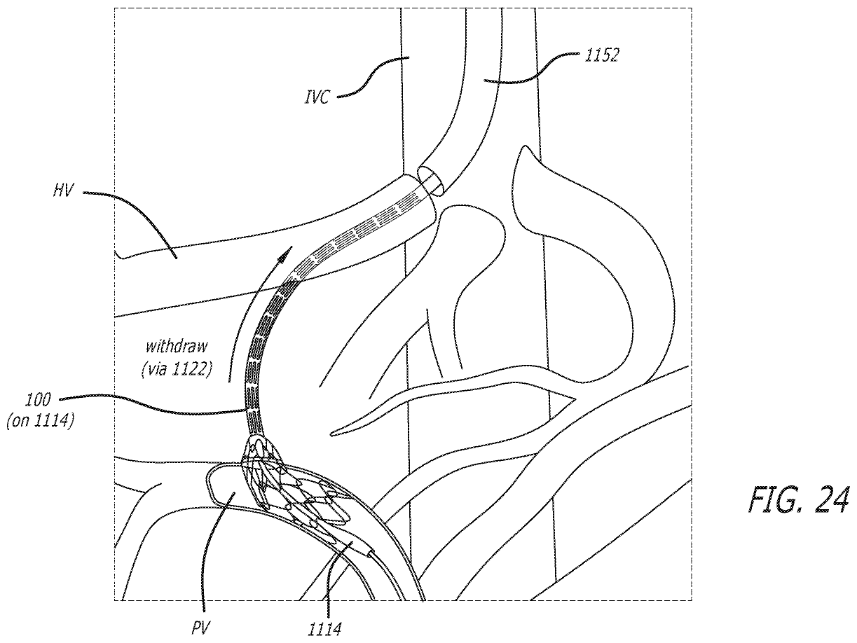

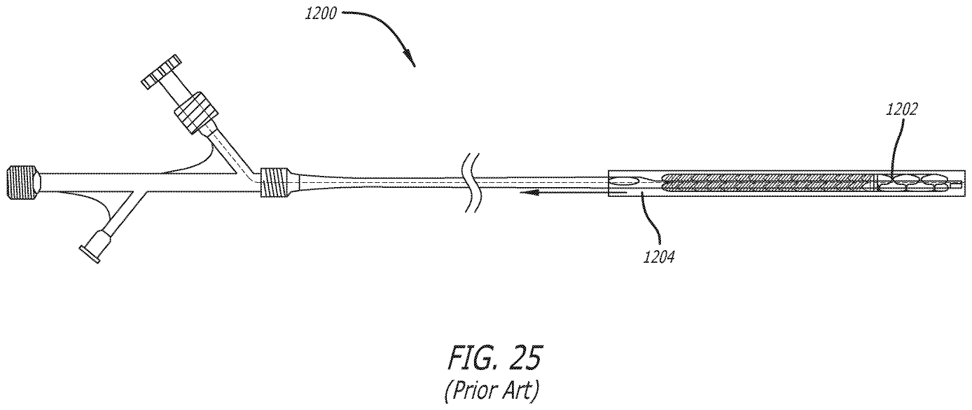

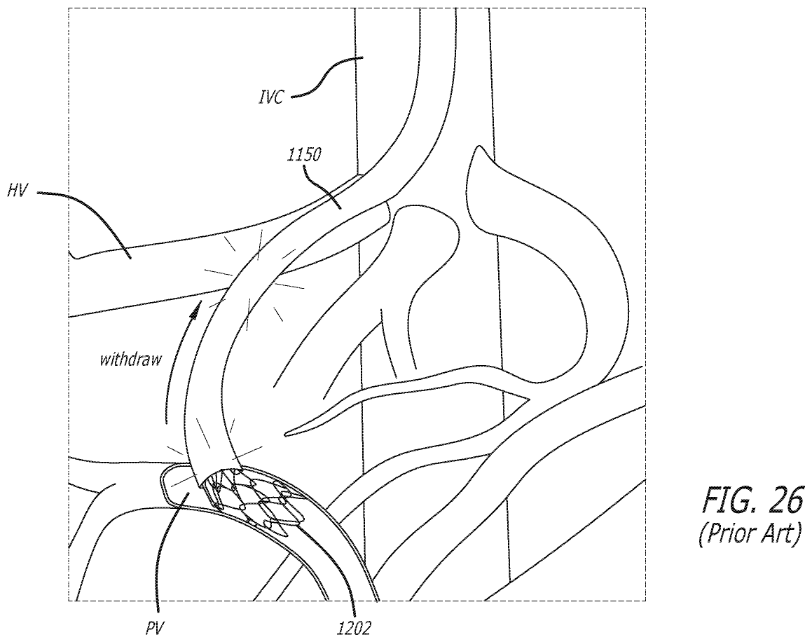

Advantageously, the delivery system 1110 deploys the endoluminal prosthesis 100 in a simpler and less traumatic procedure than that for prior-art endoluminal prostheses. This is shown by way of FIGS. 23-26, wherein: FIG. 23 illustrates the delivery system 1110 for delivering the endoluminal prosthesis 100 in accordance with some embodiments; FIG. 24 illustrates deploying the endoluminal prosthesis 100 with the delivery system 1110 in accordance with some embodiments; FIG. 25 illustrates a prior-art delivery system 1200 for delivering a prior-art endoluminal prosthesis 1202; and FIG. 26 illustrates deploying the prior-art endoluminal prosthesis 1202 with the prior-art delivery system 1200.

Beginning with the procedure for delivering prior-art endoluminal prostheses, the shunt created via standard portal vein access technique is maintained by a 10-Fr introducer sheath 1150 extending from the hepatic vein (“HV”), across the liver parenchyma, to well within the portal vein (“PV”) as shown in FIG. 26. It is to the introducer sheath 1150 that the prior-art endoluminal prosthesis 1202 is initially introduced from its as-packaged constraining tube 1204, within the introducer sheath 1150 that the prior-art endoluminal prosthesis 1202 is subsequently constrained while being advanced, and from the distal end of the introducer sheath 1150 that the prior-art endoluminal prosthesis 1202 is ultimately deployed upon withdrawal of the introducer sheath 1150 from the shunt. Not only does this ultimately require a larger device than needed to maintain the shunt, as below, but transferring between constraining means for constraining the prior-art endoluminal prosthesis 1202 when introducing the prior-art endoluminal prosthesis 1202 to the introducer sheath 1150 is finicky and subject to clinician error. For example, if the constraining tube 1204 is not adequately held together with a remainder of the distal portion of the prior-art delivery system 1200 while inserting it into the extracorporeal valved hub of the introducer sheath 1150, the constraining tube 1204 can slide proximally over the remainder of the distal portion of the prior-art delivery system 1200 (see arrow in FIG. 25), thereby partially deploying the prior-art endoluminal prosthesis 1202 in the valved hub of the introducer sheath 1150. Being that the valved hub of the introducer sheath 1150 has a greater inner diameter than the introducer sheath 1150 distal thereto, it can be difficult if not impossible to further advance the prior-art endoluminal prosthesis 1202 into the introducer sheath 1150 depending upon the degree of its inadvertent deployment in the valved hub. Lastly, deploying the prior-art endoluminal prosthesis 1202 from the introducer 1150 itself changes the introducer sheath 1150 from a stationary device into a working device that further traumatizes the very tissue in which the prior-art endoluminal prosthesis 1202 is to be placed.

While the delivery system 1110 might use an introducer sheath 1152 similar in size to the introducer sheath 1150, for example, 10 Fr, the introducer sheath 1152 can instead be positioned outside the shunt, such as in the hepatic vein or even in the inferior vena cava (“IVC”), which reduces trauma to the tissue around the shunt in which the endoluminal prosthesis 100 is to be placed. Even when the catheter 1114 of the delivery system 1110 extends from the hepatic vein, across the liver parenchyma, to within the portal vein for deploying the endoluminal prosthesis 100 as shown in FIG. 24, the catheter 1114 has a smaller profile than the introducer sheath 1150, thereby maintaining a reduction in trauma to the tissue around the shunt. Indeed, being that the catheter 1114 itself holds and constrains the endoluminal prosthesis 100 instead of relying upon the introducer sheath 1152 for the constraining means, the catheter 1114 can be less than or equal to 9.5 Fr, 9.0 Fr, 8.5 Fr, 8.0 Fr, 7.5 Fr, 7.0 Fr, 6.5 Fr, or 6.0 Fr to facilitate movement through the introducer sheath 1152. Lastly, regarding the catheter 1114 itself holding and constraining the endoluminal prosthesis 100, the prosthesis-holding and prosthesis-constraining sections of the endoluminal prosthesis 100 make it such that there is no need for transferring the endoluminal prosthesis 100 between constraining means for constraining it like that set forth above for the prior-art endoluminal prosthesis 1202, which transferring is finicky and subject to clinician error. Indeed, the catheter 1114 with the endoluminal prosthesis 100 held and restrained thereon merely needs to be inserted into the introducer sheath 1152, thereby reducing clinician error compared to the existing procedure for prior-art endoluminal prostheses.

In view of the foregoing, a method for delivering the endoluminal prosthesis 100 includes obtaining the delivery system 1110 including the catheter 1114 that independently holds and constrains the endoluminal prosthesis 100; advancing the introducer sheath 1152 to the hepatic vein by way of the inferior vena cava; creating a shunt extending from the hepatic vein, across the liver parenchyma, and into the portal vein; advancing the catheter 1114 from the introducer sheath 1152, across the shunt, and well into the portal vein while the distal end of the introducer sheath 1152 is positioned outside the shunt; and deploying the endoluminal prosthesis 100 across the shunt while withdrawing the catheter 1114 from the shunt, thereby deploying the endoluminal prosthesis 100 in a simpler and less traumatic procedure than that for prior-art endoluminal prostheses.

In some embodiments, the catheter 1114 includes the prosthesis-holding portion at the distal end of the catheter 1114 as well as the prosthesis-constraining sheath that covers the prosthesis-holding portion, thereby holding and constraining the endoluminal prosthesis 100 with the catheter 1114 alone until deploying the endoluminal prosthesis 100 across the shunt.

In some embodiments, the method further includes operating the thumbwheel 1122 extending from the handle 1112 of the delivery system 1110 to proximally pull the prosthesis-constraining sheath away from the endoluminal prosthesis 100 and uncover it while withdrawing the catheter 1114 from the shunt.

In some embodiments, the method further includes actuating the second release mechanism 1124, thereby allowing the endoluminal prosthesis 100 to be partially uncovered for partial deployment of the endoluminal prosthesis 100 in the portal vein while operating the thumbwheel 1122.

In some embodiments, the second release mechanism 1124 includes a slider in the handle 1112, wherein actuating the second release mechanism 1124 includes sliding the slider.

In some embodiments, the method further includes actuating the first release mechanism 1128, thereby allowing the endoluminal prosthesis 100 to be fully uncovered for full deployment of the endoluminal prosthesis 100 in the shunt while operating the thumbwheel 1122.

In some embodiments, the first release mechanism 1128 includes a button in the handle 1112, wherein actuating the first release mechanism 1128 includes pushing the button.

In some embodiments, the distal end of the introducer sheath 1152 is positioned in the inferior vena cava while advancing the catheter 1114 from the introducer sheath 1152, across the shunt, and well into the portal vein, thereby precluding trauma from advancing the introducer sheath 1152 through the hepatic vein, across the shunt, and well into the portal vein.

In some embodiments, the distal end of the introducer sheath 1152 is positioned in the hepatic vein but outside the shunt while advancing the catheter 1114 from the introducer sheath 1152, across the shunt, and well into the portal vein, thereby precluding trauma from advancing the introducer sheath 1152 through the hepatic vein, across the shunt, and well into the portal vein.

In some embodiments, the catheter 1114 independently holds and constrains the endoluminal prosthesis 100 without assistance from the introducer sheath 1152, thereby precluding transfer of the endoluminal prosthesis 100 between constraining means for constraining the endoluminal prosthesis 100.

In some embodiments, the introducer sheath 1152 is a 10-Fr introducer sheath.

In some embodiments, the catheter 1114 has a smaller profile than the introducer sheath 1152, thereby reducing trauma to tissue around the shunt.

Claim 1: A method for deploying an endoluminal prosthesis, comprising: obtaining a delivery system including a catheter that independently holds and constrains the endoluminal prosthesis; advancing an introducer sheath to a hepatic vein by way of an inferior vena cava; creating a shunt extending from the hepatic vein, across a liver parenchyma, and into a portal vein; advancing the catheter from the introducer sheath, across the shunt, and well into the portal vein while a distal end of the introducer sheath is positioned outside the shunt; and deploying the endoluminal prosthesis across the shunt while withdrawing the catheter from the shunt, thereby deploying the endoluminal prosthesis in a simpler and less traumatic procedure than that for prior-art endoluminal prostheses.

Claim 2: The method of claim 1, wherein the catheter includes a prosthesis-holding portion at a distal end of the catheter as well as a prosthesis-constraining sheath that covers the prosthesis holding portion, thereby holding and constraining the endoluminal prosthesis with the catheter alone until deploying the endoluminal prosthesis across the shunt.

Claim 3: The method of claim 2, further comprising operating a thumbwheel extending from a handle of the delivery system to proximally pull the prosthesis-constraining sheath away from the endoluminal prothesis and uncover it while withdrawing the catheter from the shunt.

Claim 4: The method of claim 3, further comprising actuating a second release mechanism, thereby allowing the endoluminal prosthesis to be partially uncovered for partial deployment of the endoluminal prothesis in the portal vein while operating the thumbwheel.

Claim 5: The method of claim 4, wherein the second release mechanism includes a slider in the handle, wherein actuating the second release mechanism includes sliding the slider.

Claim 6: The method of claim 4, further comprising actuating a first release mechanism, thereby allowing the endoluminal prosthesis to be fully uncovered for full deployment of the endoluminal prothesis in the shunt while operating the thumbwheel.

Claim 7: The method of claim 6, wherein the first release mechanism includes a button in the handle, wherein actuating the first release mechanism includes pushing the button.

Claim 8: The method of claim 1, wherein the distal end of the introducer sheath is positioned in the inferior vena cava while advancing the catheter from the introducer sheath, across the shunt, and well into the portal vein, thereby precluding trauma from advancing the introducer sheath through the hepatic vein, across the shunt, and well into the portal vein.

Claim 9: The method of claim 1, wherein the distal end of the introducer sheath is positioned in the hepatic vein but outside the shunt while advancing the catheter from the introducer sheath, across the shunt, and well into the portal vein, thereby precluding trauma from advancing the introducer sheath through the hepatic vein, across the shunt, and well into the portal vein.

Claim 10: The method of claim 1, wherein the catheter independently holds and constrains the endoluminal prosthesis without assistance from the introducer sheath, thereby precluding transfer of the endoluminal prosthesis between constraining means for constraining the endoluminal prosthesis.

Claim 11: The method of claim 1, wherein the introducer sheath is a 10-Fr introducer sheath.

Claim 12: The method of claim 1, wherein the catheter has a smaller profile than the introducer sheath, thereby reducing trauma to tissue around the shunt.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A stent graft, comprising:
a framework including a helical section, a woven section, and a transitional section between the helical section and the woven section;
a plurality of radiopaque markers distributed about an end portion of the helical section opposite the transitional section, an end portion of the woven section opposite the transitional section, or both the end portion of the helical section and the end portion of the woven section;
a graft including an inner graft member and an outer graft member encapsulating at least the helical section of the framework; and
a constrictive sleeve over the outer graft member of the graft, the constrictive sleeve configured to resist radial expansion of the stent graft over a length of the stent graft commensurate with the constrictive sleeve.

2. The stent graft of claim 1, wherein the framework consists of a single wire and three wire welds, the wire formed into each section of the helical section, the woven section, and the transitional section of the framework, and the three wire welds respectively distributed about the helical section, the woven section, and the transitional section of the framework to maintain a topology of the framework.

3. The stent graft of claim 2, wherein an end portion of the wire in the end portion of the helical section opposite the transitional section is welded to an internal portion of the wire in the end portion of the helical section of the framework.

4. The stent graft of claim 3, wherein the end portion of the wire in the end portion of the helical section opposite the transitional section of the framework includes a rounded end of the wire, thereby obviating any potentially traumatic edges in the end portion of the helical section opposite the transitional section.

5. The stent graft of claim 2, wherein an end portion of the wire in the transitional section is welded to an internal portion of the wire in the transitional section of the framework.

6. The stent graft of claim 5, wherein the end portion of the wire in the transitional section of the framework includes a rounded end of the wire, thereby obviating any potentially traumatic edges in the transitional section.

7. The stent graft of claim 2, wherein a looped portion of the wire in the end portion of the woven section opposite the transitional section is welded to an internal portion of the wire in the end portion of the woven section of the framework.

8. The stent graft of claim 2, wherein the wire consists essentially of nitinol.

9. The stent graft of claim 1, wherein the end portion of the helical section opposite the transitional section of the framework includes three radiopaque markers crimped onto the framework approximately equidistant from each other around the end portion of the helical section.

10. The stent graft of claim 1, wherein the end portion of the woven section opposite the transitional section of the framework includes three radiopaque markers crimped onto the framework approximately equidistant from each other around the end portion of the helical section.

11. The stent graft of claim 1, wherein the radiopaque markers consist essentially of tantalum.

12. The stent graft of claim 1, further comprising a radiopaque marker band around the transitional section of the framework, the radiopaque marker band consisting essentially of tungsten, which is printed as discrete quadrilaterals on a ring member disposed between the framework and the outer graft member of the graft.

13. The stent graft of claim 1, wherein each graft member of the inner graft member and the outer graft member independently consists essentially of expanded polytetrafluoroethylene (“ePTFE”), optionally, impregnated with carbon for reduced thrombogenicity.

14. The stent graft of claim 13, wherein the inner graft member consists essentially of ePTFE impregnated with carbon and the outer graft member consists essentially of ePTFE without carbon impregnated therein.

15. The stent graft of claim 1, wherein the constrictive sleeve is coupled to the graft exclusively by an interference fit.

16. The stent graft of claim 1, wherein the constrictive sleeve has a length shorter than that of the graft, the constrictive sleeve is approximately centered over the graft, and both the end portion of the helical section opposite the transitional section and an end portion of the helical section proximate the transitional section extending through the end portion of the woven section opposite the transitional section are flared in a ready-to-deploy state of the stent graft.

17. The stent graft of claim 1, wherein the constrictive sleeve includes an unsintered ePTFE tube, a first layer of unsintered ePTFE membrane, and a second layer of unsintered ePTFE membrane.

18. The stent graft of claim 17, wherein an unsintered ePTFE membrane sheet forms the first layer of unsintered ePTFE membrane and the second layer of unsintered ePTFE membrane by wrapping the unsintered ePTFE membrane sheet twice around the unsintered ePTFE tube.

19. The stent graft of claim 1, wherein the constrictive sleeve is configured to resist radial expansion of the stent graft over the length of the stent graft commensurate with the constrictive sleeve until radially expanded in vivo with an elongate balloon.

20. A stent graft, comprising:
a framework including a helical section, a woven section, and a transitional section between the helical section and the woven section, wherein:
the framework consists of a single nitinol wire and three wire welds;
the wire is formed into each section of the helical section, the woven section, and the transitional section of the framework; and
the three wire welds are respectively distributed about the helical section, the woven section, and the transitional section of the framework to maintain a topology of the framework;
three tantalum markers crimped onto the framework approximately equidistant from each other around an end portion of the helical section opposite the transitional section of the framework;
another three tantalum markers crimped onto the framework approximately equidistant from each other around an end portion of the woven section opposite the transitional section of the framework;
a graft including an inner graft member and an outer graft member encapsulating at least the helical section of the framework, wherein:
the inner graft member consists essentially of expanded polytetrafluoroethylene (“ePTFE”) impregnated with carbon for reduced thrombogenicity; and
the outer graft member consists essentially of ePTFE without carbon impregnated therein; and
a tungsten radiopaque marker band around the transitional section of the framework, wherein the tungsten radiopaque marker band is printed as discrete squares on a ring member disposed between the framework and the outer graft member of the graft; and
a constrictive sleeve shorter in length than the graft, approximately centered over the graft, and coupled to the outer graft member of the graft exclusively by an interference fit, wherein:
the constrictive sleeve includes an unsintered ePTFE tube, a first layer of unsintered ePTFE membrane, and a second layer of unsintered ePTFE membrane; and
the constrictive sleeve is configured to resist radial expansion of the stent graft over the length of the stent graft commensurate with the constrictive sleeve until radially expanded in vivo with an elongate balloon.
